(19) United States
(12) Patent Application Publication
(10) Pub. No.: US 2022/0256794 A1
(43) Pub. Date: Aug. 18, 2022

(54) TERPENE SYNTHASES AND TRANSPORTERS

(71) Applicant: The State of Israel, Ministry of Agriculture & Rural Development, Agricultural Research Organization, Rishon-LeZion (IL)

(72) Inventors: Shahar COHEN, Rishon-LeZion (IL); Rachel DAVIDOVICH-RIKANATI, Alonei Abba (IL); Einat BAR, Kfar Baruch (IL); Efraim LEWINSOHN, Timrat (IL); Adi DORON-FAIGENBOIM, Ramat-HaSharon (IL); Faris SALAMA, Qalansuwa (IL); Daniel HASSON, Tel Aviv (IL); Shahar SARUBERG, Mahanaim (IL); Yelena YESELSON, Ashdod (IL); Dalia WOLF, Nes Ziona (IL); Guy POLTURAK, Norwich (GB); Maxim ITKIN, Kibbutz HaOgen (IL); Arthur A. SCHAFFER, Hashmonaim (IL)

(73) Assignee: The State of Israel, Ministry of Agriculture & Rural Development, Agricultural Research Organization, Rishon-LeZion (IL)

(21) Appl. No.: 17/609,479

(22) PCT Filed: May 8, 2020
(86) PCT No.: PCT/IL2020/050505

§ 371 Date: Nov. 8, 2021

Related U.S. Application Data
(60) Provisional application No. 62/844,767, filed on May 8, 2019.

(51) Int. Cl. A01H 1/00; C12N 9/88; C12P 5/02; C12P 7/04; C12P 5/00; A01H 5/12; A01H 6/28

(52) U.S. Cl. CPC A01H 1/101; C12N 9/88; C12Y 402/03; A01H 6/28; C12P 7/04; C12P 5/002; A01H 5/12; C12P 5/026

(57) ABSTRACT

A plant comprising a genome having an introgression which comprises a polynucleotide sequence encoding a polypeptide having a terpene synthase activity or a transporter activity, the introgression comprising allelic variation(s) as compared to a genome of a recurrent parent of the plant, is disclosed. Organisms comprising a genome having been genetically modified to express a polypeptide having a terpene synthase activity or a transporter activity are also disclosed. Methods of modulating terpene synthesis or transport of metabolites in organisms, methods of producing plants having a terpene synthase activity of interest, having a terpene profile of interest or having a transporter activity of interest are disclosed. Methods of producing a terpene of interest are also disclosed.

### RELATED APPLICATION/S

This application claims the benefit of priority of U.S. Provisional Patent Application No. 62/844,767 filed on May 8, 2019, the contents of which are incorporated herein by reference in their entirety.

### SEQUENCE LISTING STATEMENT

The ASCII file, entitled 82332SequenceListing.txt, created on May 7, 2020, comprising 644,305 bytes, submitted concurrently with the filing of this application is incorporated herein by reference.

### FIELD AND BACKGROUND OF THE INVENTION

The present invention, in some embodiments thereof, relates to terpene synthases and transporters and, more particularly, but not exclusively, to the expression of terpene synthases and transporters for modulating expression of secondary metabolites in plants of interest.

Plants produce a large number of secondary metabolites, which are classified into several groups according to their biosynthetic routes and structural features [Yazaki K., Transporters of secondary metabolites. Curr. Opin. Plant Biol. (2005) 8:301-307]. Currently, more than 200,000 secondary metabolites have been identified. Among these compounds, various volatile organic compounds (VOCs) and cannabinoids have recently become subjects of great interest as they tend to encompass valuable pharmaceutical properties. Plant VOCs are chemically diverse and are mainly represented by terpenoids, fatty acid derivatives, benzenoids, and phenylpropanoids.

Cannabis plants have long been used in drug and industrial applications (e.g. for fiber, seed, seed oils, and medical purposes). Cannabis plants produce cannabinoids, terpenes, and other compounds. Cannabinoids, the most studied group of secondary metabolites in cannabis, are a large family of approximately 150 active compounds that activate cannabinoid receptors in cells and alter neurotransmitter release in the brain.

Terpenes and terpenoids, a large and diverse class of VOCs, are produced by a variety of plants and are typically associated with odor and flavor. Terpenes of cannabis are classically simple monoterpenes (e.g. D-limonene, β-myrcene, α- and β-pinene, terpinolene and linalool) and sesquiterpenes (e.g. β-caryophyllene and α-humulene) derived from two and three isoprene units, respectively. Terpenoids (isoprenoids) are derived from five-carbon isoprene units. As cannabinoids are odorless, terpenes and terpenoids are responsible for the unique odor of cannabis, and each cannabis variety has a slightly different terpene/terpenoid profile that can be used as a tool for identification of cannabis varieties or geographical origins of samples [Hillig, Biochem System and Ecology (2004) 875-891]. The usefulness of individual mono- and sesquiterpenes in Cannabis has been extensively reviewed [Andre et al. Frontiers in Plant Science (2016) 7:19, and Russo E B, British J Pharmacol. (2011), 163: 1344-1364].

Cannabis plants produce and accumulate a terpene-rich resin in glandular trichomes, which are most abundant on the surface of female inflorescences. Certain cannabis varieties are rich in terpenes, e.g. in Cannabis sativa L. plants terpenes comprise up to 3-5% of the dry-mass of the female inflorescence. Some terpenes found in Cannabis sativa are relatively well known for their potential in biomedicine and have been used in traditional medicine (e.g. as neuroprotective, antidepressant, anti-inflammatory and anti-cancer agents), while others are yet to be studied in detail (discussed in Tarmo Nuutinen, European Journal of Medicinal Chemistry. (2018) 157:198-228). Moreover, some studies have indicated that terpenes and terpenoids can interact with lipid membranes, with ion channels, with a variety of different receptors (including both G-protein coupled odorant and neurotransmitter receptors) and with enzymes. Thus, terpenes and terpenoids produced by cannabis plants may alter the permeability of cell membranes and allow in either more or less THC, other terpenes and terpenoids can affect serotonin and dopamine chemistry as neurotransmitters, and some terpenes and terpenoids are capable of absorption through human skin and passing the blood brain barrier (U.S. Pat. No. 9,642,317).

Terpenoids are mainly synthesized in two metabolic pathways: mevalonic acid/MEV pathway (i.e. the mevalonate-dependent pathway, i.e. HMG-CoA reductase pathway) which takes place in the cytosol and has been suggested to be responsible for synthesis of sesquiterpenes (C15), and MEP/DOXP pathway (i.e. the 2-C-methyl-D-erythritol 4-phosphate/1-deoxy-D-xylulose 5-phosphate pathway, non-mevalonate pathway, or mevalonic acid-independent pathway) which takes place in plastids and has been suggested to be responsible for synthesis of hemi-(C5), mono-(C10), and diterpenes-(C20). Terpene synthases (including monoterpene synthases, diterpene synthases, and sesquiterpene synthases) are responsible for synthesis of a large number of terpenes in plants using substrates provided by these two metabolic pathways, in particular, from geranyl diphosphate (GPP), farnesyl diphosphate (FPP), geranylgeranyl diphosphate (GGPP), and any combination of two or more of these. Generally, the terpene synthase gene family of plants is classified into 6 subfamilies, referred to as a, b, c, d, e/f and g. The generalization is that the b subfamily encodes enzymes that catalyze the synthesis of cyclic monoterpenes, the g family of non-cyclic monoterpenes, and the a family catalyzes sesquiterpene synthesis. The additional families comprise diterpene and larger terpene synthases, as well as monocot genes for monoterpene synthases.

The TPS family of Cannabis has been only partially described and characterized. Recent descriptions of draft genomes and transcriptomes for the hemp-type “Finola” variety and the marijuana-type “Purple Kush” variety have been made publically available [van Bakel H et al., Genome Biology. (2011) 12: p.R102]. Booth et al. [Booth J K et al., PLoS ONE (2017) 12(3): e0173911] carried out a transcriptome analysis of trichomes of the cannabis hemp variety ‘Finola’ revealing sequences of all stages of terpene biosynthesis. Specifically, Booth et al. identified nine cannabis terpene synthases (CsTPS) in subfamilies TPS-a and TPS-b in addition to four TPS genes derived from the Purple Kush genome and transcriptome assembly (van Hakel el al., above). Functional characterization of these 13 putative mono- and sesqui-TPS, indicated products which collectively comprised most of the terpenes of ‘Finola’ resin, including β-myrcene, (E)-β-ocimene, (−)-limonene, (+)-α-pinene, β-caryophyllene, and α-humulene.

Plants producing secondary metabolites must take great care in their production and storage in order to avoid self-toxicity effects. Plants accomplish this task via numerous detoxification mechanisms in order to eliminate or modify toxic compounds, such as their excretion into extracellular compartments, sequestration into vacuoles, biosynthesis in extracellular compartments and their modification into inactive forms. Secondary metabolites are transported actively within the plant tissue by energy-dependent, molecule-specific transporters, particularly of the ATP-binding Cassette transporter family, referred to as ABC transporters.

The ABC superfamily of proteins is ubiquitous in all kingdoms of life, composed mainly of primary active transporters, which comprise nucleotide binding domains (NBDs) and transmembrane domains (TMD). While the TMDs bind and translocate substrates across membranes, the NBDs hydrolyze ATP and provide the energy for active transport. The ABC family comprises a large number (more than 120) of putative ABC transporter encoding genes in many plants. Plant ABC transporters are divided, based on protein sequence similarities, into eight distinct subfamilies, named alphabetically from ABCA to ABCH. Among these, the ABCG subfamily is found only in plants and fungi, and consists of two major types of proteins; half-size transporters (ABCG-11/WBC11-white-brown complex), and full-size transporters (earlier known as plant pleiotropic drug-resistant (PDR)-like subfamily). In plants, PDR transporters are reported to be involved in varieties of biological functions, including terpenoids transport. Another subfamily of the ABC transporters is the ABCB subfamily, which include the biologically important multidrug resistant (MDR) protein and the transporter associated with antigen processing (TAP) complex. Current understanding of relations between ABC transporters and their substrate group of secondary metabolites, mainly alkaloids, phenolic compounds, terpenoids and wax, cuticle and suberin-related compounds, has been recently summarized [Shitan N., Bioscience, Biotechnology, and Biochemistry (2016) 80:1283-1293].

An additional family of transporter proteins is the Peptide Transporter (PTR) family, which consists of 53 nitrate transporter 1/peptide transporter (NPF) (NRT1/PTR FAMILY) members in Arabidopsis. Plant NPF proteins can also transport a large variety of substrates, including dipeptides, nitrate, nitrite, chloride, glucosinolates, and amino-acids, as well as several plant hormones [Corratgé-Faillie C. and Lacombe B., Journal of Experimental Botany (2017) 68:3107-3113].

Additional background art includes U.S. Patent Application no. 20080281135 relating to methods for producing terpenes of interest in plants having glandular trichomes (e.g. cannabis plants).

### SUMMARY OF THE INVENTION

According to an aspect of some embodiments of the present invention there is provided a plant comprising a genome having an introgression which comprises a polynucleotide sequence encoding a polypeptide having a terpene synthase activity, the polypeptide comprising an amino acid sequence selected from the group consisting of SEQ ID NO: 1-45, the introgression comprising allelic variation(s) as compared to a genome of a recurrent parent of the plant.

According to an aspect of some embodiments of the present invention there is provided a method of producing a plant having a terpene synthase activity of interest, the method comprising: (a) crossing a plant which comprises a polynucleotide sequence encoding a polypeptide having a terpene synthase activity, the polypeptide comprising an amino acid sequence selected from the group consisting of SEQ ID NO: 1-45 with a plant of interest, the plant of interest being a recurrent parent; and (b) selecting from a progeny of the crossing a plant having the terpene synthase activity of interest.

According to an aspect of some embodiments of the present invention there is provided a method of producing a plant having a terpene profile of interest, the method comprising: (a) crossing the plant of some embodiments of the invention, with a plant of interest; and (b) selecting from a progeny of the crossing a plant having the terpene profile of interest.

According to an aspect of some embodiments of the present invention there is provided an organism comprising a genome having been genetically modified to express a polypeptide having a terpene synthase activity, the polypeptide being at least 95% identical to an amino acid sequence selected from the group consisting of SEQ ID NO: 1-45.

According to an aspect of some embodiments of the present invention there is provided a method of modulating terpene synthesis in an organism, the method comprising over-expressing within at least one cell of the organism a polypeptide having a terpene synthase activity, the polypeptide being at least 95% identical to an amino acid sequence selected from the group consisting of SEQ ID NO: 1-45, thereby modulating terpene synthesis in the organism.

According to an aspect of some embodiments of the present invention there is provided an isolated polypeptide comprising an amino acid sequence at least 95% identical to the amino acid sequence selected from the group consisting of SEQ ID NOs: 1-45, wherein the polypeptide, when expressed in an organism, is capable of modulating the synthesis of a terpene of interest.

According to an aspect of some embodiments of the present invention there is provided a plant comprising a genome having an introgression which comprises a polynucleotide sequence encoding a polypeptide having a transporter activity, the polypeptide comprising an amino acid sequence selected from the group consisting of SEQ ID NO: 46-57, 116, 118, 120, 122, 124, 126, 128 and 130, the introgression comprising allelic variation(s) as compared to a genome of a recurrent parent of the plant.

According to an aspect of some embodiments of the present invention there is provided a method of producing a plant having a transporter activity of interest, the method comprising: (a) crossing the plant of some embodiments of the invention, with a plant of interest; and (b) selecting from a progeny of the crossing a plant having the transporter activity of interest.

According to an aspect of some embodiments of the present invention there is provided an organism comprising a genome having been genetically modified to express a polypeptide having a transporter activity, the polypeptide being at least 95% identical to an amino acid sequence selected from the group consisting of SEQ ID NO: 46-57, 116, 118, 120, 122, 124, 126, 128 and 130.

According to an aspect of some embodiments of the present invention there is provided a method of modulating transport of metabolites in an organism, the method comprising over-expressing within at least one cell of the organism a polypeptide having a transporter activity, the polypeptide being at least 95% identical to an amino acid sequence selected from the group consisting of SEQ ID NO: 46-57, 116, 118, 120, 122, 124, 126, 128 and 130, thereby modulating the transport of the metabolites in the organism.

According to an aspect of some embodiments of the present invention there is provided an isolated polypeptide comprising an amino acid sequence at least 95% identical to the amino acid sequence selected from the group consisting of SEQ ID NOs: 46-57, 116, 118, 120, 122, 124, 126, 128 and 130, wherein the polypeptide, when expressed in an organism, is capable of modulating transport of metabolites.

According to an aspect of some embodiments of the present invention there is provided an isolated polynucleotide encoding a polypeptide having a terpene synthase activity, the polypeptide being at least 95% identical to an amino acid sequence selected from the group consisting of SEQ ID NO: 1-45.

According to an aspect of some embodiments of the present invention there is provided an isolated polynucleotide encoding a polypeptide having a transporter activity, the polypeptide being at least 95% identical to an amino acid sequence selected from the group consisting of SEQ ID NO: 46-57, 116, 118, 120, 122, 124, 126, 128 and 130.

According to an aspect of some embodiments of the present invention there is provided a nucleic acid construct comprising a nucleic acid sequence of the polynucleotide of some embodiments of the invention, and a cis-acting regulatory element for directing expression of the nucleic acid sequence in a cell.

According to an aspect of some embodiments of the present invention there is provided an isolated cell comprising at least one exogenous polynucleotide according to of some embodiments of the invention, or construct according to some embodiments of the invention.

According to an aspect of some embodiments of the present invention there is provided a genetically modified organism comprising at least one exogenous polynucleotide according to some embodiments of the invention, or construct according to some embodiments of the invention.

According to an aspect of some embodiments of the present invention there is provided a plant generated according to the method of some embodiments of the invention.

According to an aspect of some embodiments of the present invention there is provided an organism generated according to the method of some embodiments of the invention.

According to an aspect of some embodiments of the present invention there is provided a method of producing a terpene of interest, the method comprising recovering a terpene fraction comprising the terpene of interest from the plant of some embodiments of the invention, or from the organism of some embodiments of the invention.

According to an aspect of some embodiments of the present invention there is provided a terpene containing fraction of the plant of some embodiments of the invention, or of the organism of some embodiments of the invention.

According to an aspect of some embodiments of the present invention there is provided a seed of the plant of some embodiments of the invention.

According to an aspect of some embodiments of the present invention there is provided a method of producing a plant comprising sowing the seed of some embodiments of the invention or planting a plantlet of the plant of some embodiments of the invention under conditions which allow growth of the plant.

According to an aspect of some embodiments of the present invention there is provided a pharmaceutical or cosmetic composition obtainable from the plant of some embodiments of the invention, or from the organism of some embodiments of the invention.

According to an aspect of some embodiments of the present invention there is provided a food or processed product obtainable from the plant of some embodiments of the invention, or from the organism of some embodiments of the invention.

According to some embodiments of the invention, the allelic variation(s) being in a region spanning not more than 2000 base pairs.

According to some embodiments of the invention, the plant further comprises a polynucleotide sequence encoding a polypeptide having a transporter activity, the polypeptide comprising an amino acid sequence selected from the group consisting of SEQ ID NO: 46-57, 116, 118, 120, 122, 124, 126, 128 and 130.

According to some embodiments of the invention, the selecting is effected genotypically.

According to some embodiments of the invention, the selecting is effected phenotypically.

According to some embodiments of the invention, the method further comprises backcrossing to the plant of interest.

According to some embodiments of the invention, the genome further expresses a polypeptide having a transporter activity, the polypeptide being at least 95% identical to an amino acid sequence selected from the group consisting of SEQ ID NO: 46-57, 116, 118, 120, 122, 124, 126, 128 and 130.

According to some embodiments of the invention, the method comprises introducing into at least one cell of the organism an exogenous polynucleotide encoding the polypeptide.

According to some embodiments of the invention, the introducing the exogenous polynucleotide into the at least one cell comprises transforming the polynucleotide or a construct comprising same into the at least one cell.

According to some embodiments of the invention, the introducing the exogenous polynucleotide into the at least one cell comprises subjecting the at least one cell to genome editing using artificially engineered nucleases.

According to some embodiments of the invention, the plant further comprises a polynucleotide sequence encoding a polypeptide having a terpene synthase activity, the polypeptide comprising an amino acid sequence selected from the group consisting of SEQ ID NO: 1-45.

According to some embodiments of the invention, the selecting is effected genotypically.

According to some embodiments of the invention, the selecting is effected phenotypically.

According to some embodiments of the invention, the method further comprises backcrossing to the plant of interest.

According to some embodiments of the invention, the genome further expresses a polypeptide having a terpene synthase activity, the polypeptide being at least 95% identical to an amino acid sequence selected from the group consisting of SEQ ID NO: 1-45.

According to some embodiments of the invention, the transporter comprises an ATP-binding cassette transporter (ABC) or a Peptide Transporter (PTR).

According to some embodiments of the invention, the metabolites are selected from the group consisting of cannabinoids, terpenoids, alkaloids, phenolic compounds, volatile compounds, peptides, polypeptides, carotenoids, glucosinolates, benzenoids, phenylpropanoids, neurotransmitters, anthocyanins, hormones, flavonoids, organic acids, fatty acids, fatty acids derivatives, wax, cuticle and suberin-related compounds.

According to some embodiments of the invention, the cis-acting regulatory element comprises a promoter.

According to some embodiments of the invention, the promoter is plant-expressible promoter.

According to some embodiments of the invention, the plant-expressible promoter is a trichome-specific promoter.

According to some embodiments of the invention, the terpene containing fraction of some embodiments of the invention being an extract.

According to some embodiments of the invention, the terpene containing fraction of some embodiments of the invention being an oil.

According to some embodiments of the invention, the organism is a non-human organism.

According to some embodiments of the invention, the non-human organism is selected from the group consisting of a plant, a yeast, a bacteria and an insect.

According to some embodiments of the invention, the organism is a plant or part thereof.

According to some embodiments of the invention, the seed of some embodiments of the invention being a hybrid seed.

According to some embodiments of the invention, the plant is selected from a plant family of Cannabaceae, Asteraceae, Solanaceae and Lamiaceae.

According to some embodiments of the invention, the plant is a Cannabis plant.

According to some embodiments of the invention, the plant of interest is a Cannabis plant.

According to some embodiments of the invention, the plant of interest is a Cannabis sativa plant.

According to some embodiments of the invention, the part of the plant comprises a glandular trichome or a female inflorescence.

According to some embodiments of the invention, the terpene of interest or terpene profile of interest comprises at least one monoterpene and/or at least one sesquiterpene.

According to some embodiments of the invention, the modulating comprises enhancing the expression of the terpene of interest as compared to its expression in a non-genetically modified plant.

According to some embodiments of the invention, the modulating comprises enhancing the transport of metabolites as compared to transport of metabolites in a non-genetically modified plant

According to some embodiments of the invention, the pharmaceutical or cosmetic composition of some embodiments of the invention, or food or processed product of some embodiments of the invention, being an oil.

According to some embodiments of the invention, the pharmaceutical or cosmetic composition of some embodiments of the invention, or food or processed product of some embodiments of the invention, comprising DNA of the plant or of the organism.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B are graphs illustrating the effect of transporter AG0100-1 (NRT transporter) on yeast cell growth in the presences or absence of 0.5 mM CBD during a 12 hour incubation. Cell growth was measured as turbidity at A₆₀₀. (FIG. 1A) the effect of CBD on growth of the control empty plasmid yeast, (FIG. 1B) the effect of CBD on growth of the T-AG0100-1 yeast line.

FIG. 2 is a graph illustrating the effect of different transporter genes on yeast growth in the presence of 0.5 mM CBD. Results are expressed as % growth for each line with CBD, as compared to the growth of each respective line in the absence of CBD. Results are an average and s.d. of five spectrophotometric determinations at hourly intervals between 7 and 12 hours after onset of incubation. Cell growth was measured as turbidity at A₆₀₀.

FIGS. 3A-B are graphs illustrating effect of ABC transporters on net CBD uptake into yeast cells. (FIG. 3A) CBD content in yeast cells over a three hour period following incubation with 0.5 mM CBD for the ABC transporters T-792-1, T-790-1 and T-AG2575-1 compared to the empty vector. (FIG. 3B) CBD concentration in yeast cells following a 5 hour period for the ABC transporter T-792-1, compared to the empty vector. Results are averages and s.d. of 3 replications.

FIG. 4 is a graph illustrating the effect of the NRT transporter AG0100-1 on net CBD uptake into yeast cells. Results indicate the amount of CBD present in the yeast cells following a 3 hour uptake. Results are averages and s.d. of 3 replications.

FIG. 5 is a graph illustrating the effect of the NRT transporter AG0100-1 on net CBD uptake into tobacco BY-2 cells. Results indicate the amount of CBD present in the BY-2 cells throughout a 4 hour uptake. Results are averages and s.d. of 3 replications.

FIGS. 6A-B are photographs illustrating the effect of the NRT transporter AG0100-1 on CBD-induced cell death of tobacco BY-2 cells. The increased cell death in response to CBD of the BY-2 cells expressing the NRT transporter AG0100-1 is indicated by the sharp decrease in fluorescence. Two micrographs of each treatment are presented. Top of FIG. 6B shows the fluorescence of the control BY2 cells without CBD. Top of FIG. 6A shows a small decrease in the fluorescence of the control BY2 cells in the presence of 0.5 mM CBD. Bottom of FIG. 6B shows the fluorescence of the T-AG0100-1 BY2 cells without CBD, and bottom of FIG. 6A shows the strong decrease in the fluorescence of the T-AG0100-1 BY2 cells in the presence of 0.5 mM CBD.

FIG. 7 is a graph illustrating the effect of T-790-1 ABC family B transporter on net CBD uptake into tobacco BY-2 cells. Results indicate the amount of CBD present in the BY-2 cells throughout a 5 hour uptake. Results are averages and s.d. of 3 replications.

FIGS. 8A-C are graphs illustrating the effect of different transporter genes on net uptake of terpenes limonene, caryophyllene and α-pinene into yeast cells. Results indicate the amount (ng compound/mg) of individual terpenes (FIG. 8A) alpha-pinene, (FIG. 8B) limonene and (FIG. 8C) caryophyllene present in the yeast cells following a 2 hour uptake. Values represent the average of two replications and are expressed as percent of the amount of each terpene present in the empty vector yeast.

### DESCRIPTION OF SPECIFIC EMBODIMENTS OF THE INVENTION

The present invention, in some embodiments thereof, relates to terpene synthases and transporters and, more particularly, but not exclusively, to the expression of terpene synthases and transporters for modulating expression of secondary metabolites in plants of interest.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Cannabis plants produce a large number of secondary metabolites including various volatile organic compounds (VOCs) and cannabinoids. Among these VOCs are terpenoids, fatty acid derivatives, benzenoids, and phenylpropanoids. Terpenes of cannabis are classically simple monoterpenes (e.g. D-limonene, β-myrcene, α- and β-pinene, terpinolene and linalool) and sesquiterpenes (e.g. β-caryophyllene and α-humulene) synthesized by terpene synthases (TPS). In order to avoid self-toxicity effects by the secondary metabolites, plants employ detoxification mechanisms such as their excretion into extracellular compartments, sequestration into vacuoles, biosynthesis in extracellular compartments and their modification into inactive forms. Secondary metabolites are transported actively within the plant tissue by energy-dependent, molecule-specific transporters, particularly of the ATP-binding Cassette transporter family, referred to as ABC transporters. An additional family of transporter proteins is the Peptide Transporter (PTR) family, which transport a large variety of substrates, including dipeptides, nitrate, nitrite, chloride, glucosinolates, and amino-acids, as well as several plant hormones.

While reducing the present invention to practice, the present inventors have identified full length terpene synthase genes of the a, b and g terpene synthase families in cannabis plants (see Table 1 herein below). These TPS genes were shown to be expressed in the inflorescences of four different varieties of Cannabis sativa, as well as in the enriched trichome fraction of one variety (see Table 1 herein below).

The present inventors have further identified full length ATP-binding cassette transporter (ABC) and Peptide Transporter (PTR) gene families in cannabis plants (see Table 7, below). These ABC and PTR genes were shown to be expressed in the inflorescences of four different varieties of Cannabis sativa, as well as in the enriched trichome fraction of one variety (see Table 10, herein below).

The present inventors therefore propose that the newly identified TPS, ABC and PTR genes may be used in breeding of new plant varieties, such as plants comprising higher production of terpenes (i.e. via expression of TPS genes) and/or plants devoid of self-toxicity (i.e. via expression of ABC and PTR genes). The presently identified genes may be further utilized for expression of secondary metabolites (e.g. terpenes) and avoidance of self-toxicity in other organisms (e.g. bacteria, yeast and animals).

Thus, according to one aspect of the present invention there is provided a plant comprising a genome having an introgression which comprises a polynucleotide sequence encoding a polypeptide having a terpene synthase activity, the polypeptide comprising an amino acid sequence selected from the group consisting of SEQ ID NO: 1-45, the introgression comprising allelic variation(s) in compared to a genome of a recurrent parent of the plant.

According to another aspect of the present invention there is provided a plant comprising a genome having an introgression which comprises a polynucleotide sequence encoding a polypeptide having a transporter activity, the polypeptide comprising an amino acid sequence selected from the group consisting of SEQ ID NO: 46-57, 116, 118, 120, 122, 124, 126, 128 and 130, the introgression comprising allelic variation(s) in compared to a genome of a recurrent parent of the plant.

The term “organism” refers to any prokaryotic or eukaryotic organism.

Prokaryotic organisms include, but are not limited to, bacteria of the species Bacillus, Escherichia (e.g. Escherichia coli), Lactobacillus, Corynebacterium, Acetobacter, Acinetobacter and Pseudomonas.

Eukaryotic organisms include single- and multi-cellular organisms. Single cell eukaryotic organisms include, but are not limited to, yeast, protozoans, slime molds and algae. Multi-cellular eukaryotic organisms include, but are not limited to, animals (e.g. insects, invertebrates, nematodes, birds, fish, reptiles and crustaceans), plants, fungi and algae (e.g. brown algae, red algae, green algae).

According to one embodiment, the organism is not a human being.

Exemplary eukaryotic organisms include, but are not limited to, plants (as discussed below), algae (e.g. of the species Cryptista, Chloromonadophyceae, Xanthophyceae, Crypthecodinium, Chrysophyta, Bacillariophyta, Phaeophyta, Rhodophyta, Chlorophyta, Haptophyta, Cryptista, Euglenozoa, Dinozoa, Chlorarachniophyta), yeast (e.g. of the species Saccharomyces, Kluyveromycesm Candida, Pichia, Cryptococcus, Debaromyces, Hansenula, Saccharomycecopsis, Saccharomycodes, Schizosaccharomyces, Wickerhamia, Debayomyces, Hanseniaspora, Kloeckera, Zygos accharomyces, Ogataea, Kuraishia, Komagataella, Metschnikowia, Williopsis, Nakazawaea, Torulaspora, Bullera, Rhodotorula, Willopsis and Sporobolomyces), fungi (e.g. of the species Aspergillus, Penicillium, Rhizopus, Fusarium, Fusidium, Gibberella, Mucor, Mortierella, Trichoderma) and insects (as discussed below).

According to a specific embodiment, the organism is an insect.

The term insect refers to an insect at any stage of development, including an insect nymph and an adult insect. Non-limiting examples of insects include insects selected from the orders Coleoptera, Diptera (e.g. Drosophila), Hymenoptera, Lepidoptera, Mallophaga, Homoptera, Hemiptera, Orthoptera, Thysanoptera, Dermaptera, Isoptera, Anoplura, Siphonaptera, Trichoptera, Drosophilidae, Tephritidae, Pentatomidae, etc., particularly Hemiptera.

According to a one embodiment, the organism comprises a cell.

According to a one embodiment, the cell is a bacterial cell, a yeast cell, or a cell of an animal (e.g. insect cell).

The term “plant” as used herein encompasses whole plants, a grafted plant, ancestors and progeny of the plants and plant parts, including seeds, shoots, stems, roots, rootstock, scion, and plant cells, tissues and organs. The plant may be in any form including suspension cultures, embryos, meristematic regions, callus tissue, leaves, gametophytes, sporophytes, pollen, and microspores.

According to a specific embodiment, the plant is a plant cell e.g., plant cell in an embryonic cell suspension.

According to a specific embodiment, the plant comprises a plant or a plant cell generated by the method of some embodiments of the invention.

According to one embodiment, the part of the plant comprises a glandular trichome or a female inflorescence.

According to one embodiment, the plant is a wild-type plant.

According to one embodiment, the plant is non-transgenic.

According to one embodiment, the plant is transgenic.

According to one embodiment, the plant is genetically modified (GMO).

According to one embodiment, the plant is non-genetically modified (non-GMO).

According to one embodiment, the plant is a Cannabis plant.

Cannabis is a genus of flowering plants in the family Cannabaceae that includes three different species, Cannabis sativa, Cannabis indica and Cannabis ruderalis. The term Cannabis encompasses wild type Cannabis and also variants thereof, including cannabis chemovars which naturally contain different amounts of the individual cannabinoids. For example, some Cannabis strains have been selectively bred to produce high or low levels of THC and/or CBD and other cannabinoids (as discussed below). Accordingly, Cannabis cultivars that are rich in THC and/or CBD can be used in accordance with the present teachings.

For example, breeders are developing CBD-rich strains, as reported in Good, Alastair (26 Oct. 2010). “Growing marijuana that won't get you high”. The Daily Telegraph (London). Other CBD-reach strains are available from Tikun Olam that developed a strain of the plant which has only cannabidiol as an active ingredient, and no detectable levels of THC, providing some of the medicinal benefits of cannabis without the psychotrophic effects. Avidekel, a cannabis strain that contains 15.8% CBD and less than 1% THC can also be used according to the present teachings. Alternatively, strains of cannabis containing higher levels of THC than levels of (or no) CBD may be desirable for treating certain medical conditions, such as, for example, conditions causing chronic pain.

According to one embodiment, the Cannabis plant is a wild-type plant.

According to one embodiment, the Cannabis plant is non-transgenic.

According to one embodiment, the Cannabis plant is transgenic.

According to one embodiment, the Cannabis plant is genomically edited.

According to one embodiment, the Cannabis plant is Cannabis sativa (C. sativa).

According to one embodiment, the Cannabis plant is hemp.

Additional plants that may be useful in the methods of the invention include all plants which belong to the superfamily Viridiplantee, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Cannabaceae, Cannabis indica, Cannabis, Cannabis sativa, Hemp, industrial Hemp, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalypfus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago sativa, Metasequoia glyptostroboides, Moraceae, Musa sapientum, banana, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phornmium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhamnaceae, Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, trees. Alternatively algae and other non-Viridiplantae can be used for the methods of some embodiments of the invention.

According to a specific embodiment, the plant is a Humulus lupulus or a Trema orientalis.

According to a specific embodiment, the plant belongs to the buckthorn family (Rhamnaceae).

According to a specific embodiment, the plant is a Ziziphs jujuba.

According to a specific embodiment, the plant belongs to the family Moraceae.

According to a specific embodiment, the plant is a Monus notabilis.

According to a specific embodiment, the plant belongs to the Vitis family.

According to a specific embodiment, the plant is a Vitis vinmfera.

According to one embodiment, the plant is a tobacco plant.

The term “seed” as used herein refers to a flowering plant's unit of reproduction, capable of developing into another such plant.

According to one embodiment, the seed is a hybrid seed.

As used herein, the term “hybrid” means any offspring of a cross between two genetically unlike individuals, more preferably the term refers to the cross between two breeding lines which will not reproduce true to the parent from seed.

As used herein, the term “terpenes” refers to hydrocarbon compounds constructed from one or more five-carbon isoprene units which are combined to produce a diversity of skeletons. The isoprene units are typically connected in a head-to-end manner.

As used herein, the term “terpenoids”, also commonly referred to as isoprenoids, refer to terpene derivatives or analogs. Typically terpenoids are modified terpenes containing additional functional groups. Terpene derivatives/analogs include, but are not limited to, alcohols, ketones, aldehydes, ethers, acids, hydrocarbons without an oxygen functional group.

For the sake of being brief, throughout the application, the term “terpenes” is used to also refer to “terpenoids” and vice versa.

Terpenes and terpenoids comprise many volatile compounds, especially from the monoterpene and sesquiterpene subgroups, however, they may be further modified by conjugation to larger moieties such as sugar residues, which usually renders them non-volatile.

As used herein, the phrase “terpene profile of interest” refers to the expression of any one or combination of terpenes or terpenoids as discussed herein.

Determination of a terpene profile of interest may be carried out using any method known in the art, such as by gas chromatograph (GC), thin layer chromatography (TLC), gas chromatography coupled to mass spectrometer (GC-MS), liquid chromatography (LC) and liquid chromatography coupled to mass spectrometer (LC-MS), ion mobility spectrometers (IMS), high-field ion mobility spectrometers asymmetric waveform (FAIMS, high-field asymmetric waveform ion mobility spectrometry) electro-chemical sensors, electrochemical sensor arrays and colorimetric sensors.

Terpenes and terpenoids are classified according to the number of isoprene units used. The classification thus comprises the following classes: Hemiterpenes and Hemiterpenoids, 1 isoprene unit (5 carbons, i.e. 5C); Monoterpenes and Monoterpenoids, 2 isoprene units (10 carbons, i.e. 10C); Sesquiterpenes and Sesquiterpenoids, 3 isoprene units (15 carbons, i.e. 15C); Diterpenes and Diterpenoids, 4 isoprene units (20 carbons, i.e. 20C) (e.g. ginkgolides); Sesterterpenes, 5 isoprene units (25 carbons, i.e. 25C); Triterpenes and Triterpenoids, 6 isoprene units (30 carbons, i.e. 30C); Tetraterpenes and Tetraterpenoids, 8 isoprene units (40 carbons, i.e. 40C) (e.g. carotenoids) and Polyterpenes with a larger number of isoprene units.

Hemiterpenoids include, but are not limited to, Isoprene, Prenol and Isovaleric acid.

Monoterpenoids include, but are not limited to, Geranyl pyrophosphate (also known as geranyl diphosphate, GPP), Cineol, Eucalyptol, Geraniol, Limonene, Linalool, Mycrene, Nerol, Ocimene, Pinene, Terpinene and Thujene.

Sesquiterpenoids include, but are not limited to, Farnesyl pyrophosphate (FPP), Amorphadiene, Artemisinin, Aromadendrene, Bicyclogermacrene, Bisabolol, Bisabolene, Curcumene, Caryophyllene, Humulene, Farnesene and Selinene.

Diterpenoids include, but are not limited to, Geranylgeranyl pyrophosphate (GGPP), Retinol, Retinal, Phytol, Taxol, Forskolin and Aphidicolin. Another non-limiting example of a diterpene is ent-kaurene.

Sesterterpenes include, but are not limited to, Farnesyl geranyl pyrophosphate (FGPP) and Geranylfarnesyl diphosphate (FGPP).

Triterpenoids include, but are not limited to, Squalene and Lanosterol.

Tetraterpenoids include, but are not limited to, Lycopene, Carotene and Carotenoids.

Terpene and terpenoid compounds are biosynthesized from a common C5 precursor, isopentenyl pyrophosphate (IPP). This precursor is typically synthesized via the mevalonate pathway (MEV) or via the non-mevalonate pathway (or 2-C-methyl-D-erythritol 4-phosphate/1-deoxy-D-xylulose 5-phosphate (MEP/DXP) pathway) leading to the formation of IPP and its isomer dimethylallyl pyrophosphate (DMAPP). DMAPP and IPP are then condensed to generate geranyl diphosphate (GPP) which is further converted with IPP into farnesyl diphosphate (FPP). FPP is further condensed with IPP to form geranylgeranyl diphosphate (GGPP). GPP, FPP and GGPP are the precursors of monoterpenes, sesquiterpenes, diterpenes, triterpenes and carotenoids (tetraterpenoids).

Expression of the above terpene and terpenoid compounds, or combinations thereof, can be used for various purposes such as, without being limited to, enhancement of odor and/or flavor (e.g. in plants or parts thereof, e.g. flowers or fruit, food products, essential oils, cosmetics, and fragrances, as further discussed herein below), as chemotaxonomic markers in plants or parts thereof, and as pheromones in insects. The usefulness of various mono- and sesquiterpenes in Cannabis is discussed in Andre et al. Frontiers in Plant Science (2016) 7:19, and Russo E B, British J Pharmacol. (2011), 163: 1344-1364, both incorporated herein by reference.

The term “terpene synthase” or “TPS” as used herein refers to an enzyme that catalyzes the production of one or more terpenes or terpenoids from a substrate. A “functional fragment” (also referred to as “biologically active portion”) refers to a portion of a TPS capable of terpene synthesis.

According to a specific embodiment, TPS catalyzes the production of an isoprenoid compound, such as a monoterpene, sesquiterpene, diterpene, triterpene or carotenoid, from one or several precursors, in particular from geranyl diphosphate (GPP), farnesyl diphosphate (FPP), geranylgeranyl diphosphate (GGPP), and any combination of two or more of these. Generally TPSs are multi-substrate enzymes, capable of synthesizing terpenes of different chain lengths depending on corresponding substrate availability.

The term “terpene synthase activity” refers to the capability of an enzyme or complex to synthesize an isoprenoid compound (i.e. terpene or terpenoid) as determined in vivo, or in vitro, according to standard techniques (further discussed below).

Examples of terpene synthases include, without being limited to, monoterpene synthases, sesquiterpene synthases, diterpene synthases, triterpene synthases and hemiterpene synthases.

The terpene synthase may be a monoterpene synthase including, but not limited to, a geraniol synthase, a myrcene synthase, a linalool synthase (e.g. 3S-linalool synthase, R-linalool synthase), a cineol synthase (e.g. 1,8 cineol synthase), a limonene synthase (e.g. 4S-limonene synthase, R-limonene synthase), a pinene synthase (e.g. (−)-alpha-pinene synthase, (−)-beta-pinene synthase), a fenchol synthase (e.g. (−)-endo-fenchol synthase) and/or a terpineol synthase (e.g. (−)-alpha-terpineol synthase).

The terpene synthase may be a sesquiterpene synthase including, but not limited to, a farnesyl pyrophosphate synthase (FPPS), a bisabolol synthase (e.g. (+)-epi-alpha-bisabolol synthase), a germacrene synthase (e.g. germacrene A synthase, (E,E)-germacrene B synthase, germacrene C synthase, (−)-germacrene D synthase), a valencene synthase, a nerolidol synthase (e.g. (3S, 6E)-nerolidol synthase), an epi-cedrol synthase, a patchoulol synthase, a santalene synthase and/or delta-cadinene synthase.

The terpene synthase may be a diterpene synthase including, but not limited to, a geranylgeranyl pyrophosphate synthase (GGPPS) and/or an ent-kaurene synthase.

The terpene synthase may be a triterpene synthase including, but not limited to, a β-amyrin synthase.

The terpene synthase may be a hemiterpene synthase including, but not limited to, an isoprene synthase (ISPS).

The terpene synthase may be a sesterterpene synthase including, but not limited to, a farnesyl geranyl pyrophosphate synthase (FGPPS) or a geranylfarnesyl diphosphate synthase (FGPPS).

According to one embodiment, the polypeptide comprising the TPS activity comprises one terpene synthase.

According to one embodiment, the polypeptide comprising the TPS activity may produce 2, 3, 4, 5 or more different terpenes.

Exemplary polypeptides of the invention comprising TPS activity and exemplary monoterpenes and sesquiterpenes produced by expression of these TPSs (i.e. in an organism or cell) are provided in Table 6 herein below.

According to a specific embodiment, when the TPS is as set forth in SEQ ID NO: 1 it is capable of producing (Z)-beta-ocimene and/or (E)-beta-ocimene in the presence of GPP.

According to a specific embodiment, when the TPS is as set forth in SEQ ID NO: 2 it is capable of producing (Z)-beta-ocimene and/or (E)-beta-ocimene in the presence of GPP, and is capable of producing farnesene<(E,E)-alpha> in the presence of FPP.

According to a specific embodiment, when the TPS is as set forth in SEQ ID NO: 4 it is capable of producing D-limonene, alpha-pinene and/or beta pinene in the presence of GPP.

According to a specific embodiment, when the TPS is as set forth in SEQ ID NO: 8 it is capable of producing caryophyllene and/or humulene in the presence of FPP.

According to a specific embodiment, when the TPS is as set forth in SEQ ID NO: 12 it is capable of producing beta-myrcene and/or geraniol in the presence of GPP.

According to a specific embodiment, when the TPS is as set forth in SEQ ID NO: 17 it is capable of producing selina-3,7(11)-diene in the presence of FPP.

According to a specific embodiment, when the TPS is as set forth in SEQ ID NO: 18 it is capable of producing geraniol in the presence of GPP, and is capable of producing curcumene, bisabolene and/or bisabolol in the presence of FPP.

According to a specific embodiment, when the TPS is as set forth in SEQ ID NO: 23 it is capable of producing geraniol in the presence of GPP.

According to a specific embodiment, when the TPS is as set forth in SEQ ID NO: 31 it is capable of producing beta-myrcene and/or D-limonene/nerol in the presence of GPP, and is capable of producing gamma- and/or delta-selinene in the presence of FPP.

According to a specific embodiment, when the TPS is as set forth in SEQ ID NO: 33 it is capable of producing myrcene in the presence of GPP.

According to a specific embodiment, when the TPS is as set forth in SEQ ID NO: 38 it is capable of producing beta-myrcene and/or linalool in the presence of GPP, and is capable of producing caryophyllene 9 epi, aromadendrene and/or bicyclogermacrene in the presence of FPP.

According to a specific embodiment, when the TPS is as set forth in SEQ ID NO: 40 it is capable of producing beta-myrcene and/or geraniol in the presence of GPP.

According to a specific embodiment, when the TPS is as set forth in SEQ ID NO: 41 it is capable of producing eucalyptol, gamma terpinene and/or alpha thujene in the presence of GPP.

According to a specific embodiment, when the TPS is as set forth in SEQ ID NO: 36 it is capable of producing bisabolene in the presence of FPP.

The term “transporter activity” refers to the activity exerted by a transporter protein or polypeptide on a transporter substrate (molecule and ion), as determined in vivo, or in vitro, according to standard techniques (further discussed below).

According to one embodiment, a transporter activity comprises the activation of a transport dependent signal transduction pathway.

According to one embodiment, a transporter activity comprises modulation of the transport of a substrate across a membrane, e.g. cell membrane/wall.

According to one embodiment, a transporter activity comprises an interaction of a transport protein with a non-transport membrane-associated molecule.

According to one embodiment, the term “transporter activity of interest” refers to the expression of any one or combination of the transporters discussed herein.

The term “transporter” or “transporter protein” refers to a polypeptide which has at least one of the above functions. A “functional fragment” (also referred to as “biologically active portion”) refers to a portion of a transporter having at least one of the above functions.

According to a specific embodiment, the transporter protein actively translocates substrates (molecules and ions) across a membrane, e.g. the cell membrane/wall into the surrounding media.

Transporters can be grouped into families on the basis of structure, sequence homology and the molecules they transport.

According to one embodiment, the transporter is an ATP-binding cassette transporter (ABC transporter). ABC transporters are a family of membrane transporters which hydrolyze ATP and use the energy to power the transport of molecules against a concentration gradient through the membrane. ABC transporters regulate the transport of a wide variety of molecules including coating materials, supportive materials, secondary metabolites, and plant hormones. ABC transporters typically comprise one or more transmembrane domains (TMD) connected to one or more ligand binding domains (e.g. nucleotide binding domain, i.e. NBD) on either the intracellular or extracellular side of the lipid bilayer and one or more ATP binding domains on the intracellular surface. Accordingly, ABC transporters bind and use cellular adenosine triphosphate (ATP) for their specific activities. ATP transporters may be classified as half or full transporters. Full transporters may contain two transmembrane domains and two ATP binding domains and are fully functional. Half transporters contain one transdomain and one ATP binding domain and must combine with another half transporter to be fully functional. As such, a plant cell may include all or part of a transporter sufficient to confer functionality.

Exemplary polypeptides of the invention comprising transporter activity are provided in Table 8 herein below.

According to a specific embodiment, the ABC transporter is an ABC family B transporter.

According to a specific embodiment, the ABC transporter is an ABC family G transporter.

According to one embodiment, the transporter is a transporter associated with antigen processing (TAP) or the multidrug resistance efflux pump (MDR).

According to one embodiment, the transporter is a small-peptide transporter including the oligopeptide transporter (OPT) family, which can transport tetra- and penta-peptides, and the peptide transporter (PTR) family, which can transport di- and tri-peptides, as discussed in Waterworth and Bray, Annals of Botany (2006) 98: 1-8, incorporated herein by reference.

According to one embodiment, the transporter is a nitrate transporter 1/peptide transporter (NPF).

According to a specific embodiment, the transporter is a nitrate transporter (NRT).

According to one embodiment, the transporter translocates metabolites (e.g. secondary metabolites) thereby avoiding self-toxicity effects.

According to one embodiment, the metabolites comprise secondary metabolites.

According to one embodiment, the metabolites comprise cannabinoids, terpenoids, alkaloids, phenolic compounds, volatile compounds, peptides, polypeptides, carotenoids, glucosinolates, benzenoids, phenylpropanoids, neurotransmitters, anthocyanins, hormones, flavonoids, organic acids, fatty acids, fatty acids derivatives, wax, cuticle and suberin-related compounds, herbicides, fungicides, and insecticides.

According to a specific embodiment, the transporter translocates terpenes and/or cannabinoids.

Exemplary terpenes are discussed above.

Exemplary cannabinoids include, but are not limited to the acidic (caroboxylated) forms and non-acidic (decaroboxylated) forms of the following:

Tetrahydrocannabinol (THC), Cannabidiol (CBD), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV) and Cannabigerol Monomethyl Ether (CBGM) and derivatives thereof.

According to one embodiment the cannabinoids include, Alkyl phytocannabinoids, Cannabigerol (CBG)-type compounds, Cannabichromene (CBC)-type compounds, Cannabidiol (CBD)-type compounds, Thymyl-type phytocannabinoids (cannabinodiol- and cannabifuran type compounds), Tetrahydrocannabinol-type compounds, D8-tetrahydrocannabinol (D8-THC)-type compounds, D9-trans-tetrahydrocannabinol (D9-THC)-type compounds, D9-cis-Tetrahydrocannabinol-type compounds, D6a,10a Tetrahydrocannabinol and cannabitriol-type compounds, Isotetrahydrocannabinol-type compounds, Cannabicyclol (CBL)-type compounds, Cannabielsoin (CBE)-type compounds, Cannabinol (CBN)-type compounds, 8,9-Secomenthyl cannabidiols, b-Aralkyl type phytocannabinoids (phytocannabinoidlike compounds, bibenzyl cannabinoids, stiryl cannabinoids), Cannabigerol (CBG) analogues, Cannabichromene (CBC) analogues, Mentyl cannabinoids (CBD, THC) analogues.

According to one embodiment the cannabinoids include synthetic cannabinoids including, but not limited to:

Classical cannabinoids (e.g. analogs of THC based on a dibenzopyran ring), e.g. nabilone and dronabinol.

Non-classical cannabinoids, e g. cyclohexylphenols (CP) e.g. JWH-018

Aminoalkylindoles, e.g. naphthoylindoles (JWH-018), phenylacetylindoles (JWH-250), and benzoylindoles (AM-2233).

Eicosanoid synthetic and/or analogs of endocannabinoids, e.g. anandamide (N-arachidonoylethanolamide, AEA) and 2-arachidonoylglycerol (2-AG).

Natural Eicosanoid (endocannbinoids) e.g. anandamide (N-arachidonoylethanolamide, AEA) and 2-arachidonoylglycerol (2-AG).

According to one embodiment, the polypeptide comprising the transporter activity comprises one transporter.

According to one embodiment, the transporter activity comprises transport of 2, 3, 4, 5 or more different substrates.

As mentioned, the plant comprises a genome having an introgression which comprises a polynucleotide sequence encoding a polypeptide having a terpene synthase activity and/or a transporter activity.

According to one embodiment, a single plant may comprise a genome having an introgression which comprises two or more polynucleotide sequences encoding two or more polypeptides having a terpene synthase activity and/or a transporter activity.

According to one embodiment, a single plant may comprise a genome having an introgression which comprises two or more (e.g. 2, 3, 4, 5) polynucleotide sequences encoding two or more (e.g. 2, 3, 4, 5) polypeptides having a terpene synthase activity and/or a transporter activity.

As used herein, the terms “introgressing”, “introgress” and “introgressed” refer to both a natural and artificial process whereby individual genes or entire chromosomes are moved from one individual, species, variety or cultivar into the genome of another individual, species, variety or cultivar, by crossing those individuals, species, varieties or cultivars. In plant breeding, the process usually involves selfing or backcrossing to the recurrent parent to provide for an increasingly homozygous plant having essentially the characteristics of the recurrent parent in addition to the introgressed gene or trait.

The term “introgression” refers to the result of an introgression event.

The term “intercrossable”, as used herein, refers to the ability to yield progeny plants after making crosses between parent plants.

As used herein, the term “progeny” means genetic descendants or offsprings.

The terms “variety” and “cultivar” are used interchangeable herein and denote a plant with has deliberately been developed by breeding, e.g. crossing and selection, for the purpose of being commercialized.

The term “crossing” as used herein refers to the fertilization of female plants (or gametes) by male plants (or gametes). The term “gamete” refers to the haploid reproductive cell (egg or sperm) produced in plants by mitosis from a gametophyte and involved in sexual reproduction, during which two gametes of opposite sex fuse to form a diploid zygote. The term generally includes reference to a pollen (including the sperm cell) and an ovule (including the ovum). “Crossing” therefore generally refers to the fertilization of ovules of one individual with pollen from another individual, whereas “selfing” refers to the fertilization of ovules of an individual with pollen from the same individual. Crossing is widely used in plant breeding and results in a mix of genomic information between the two plants crossed one chromosome from the mother and one chromosome from the father. This will result in a new combination of genetically inherited traits. “Selfing” of a homozygous plant will usually result in a genetic identical plant since there is no genetic variation.

When referring to “crossing” in the context of achieving the introgression of a genomic region or segment, the skilled person will understand that in order to achieve the introgression of only a part of a chromosome of one plant into the chromosome of another plant, it is required that random portions of the genomes of both parental lines will be recombined during the cross due to the occurrence of crossing-over events in the production of the gametes in the parent lines. Therefore, the genomes of both parents must be combined in a single cell by a cross, where after the production of gametes from the cell and their fusion in fertilization will result in an introgression event.

The term “backcross” refers to the result of a “backcrossing” process wherein the plant resulting from a cross between two parental lines is (repeatedly) crossed with one of its parental lines, wherein the parental line used in the backcross is referred to as the recurrent parent. Repeated backcrossing results in replacement of genome fragments of the donor parent with those of the recurrent. The offspring of a backcross is designated “BCx” or “BCx population”, where “x” stands for the number of backcrosses.

The term “backcrossing” as used herein refers to the repeated crossing of a hybrid progeny back to the recurrent parents. The parental plant which contributes the gene for the desired characteristic is termed the non-recurrent or donor parent. This terminology refers to the fact that the donor parent is used one time in the backcross protocol and therefore does not recur. The parental plant to which the gene or genes from the donor parent are transferred is known as the recurrent parent as it is used for several rounds in the backcrossing protocol. In a typical backcross protocol, the original variety of interest (recurrent parent) is crossed to a second variety (donor parent) that carries the single gene of interest to be transferred. The resulting progeny from this cross are then crossed again to the recurrent parent and the process is repeated until a plant is obtained wherein essentially all of the desired morphological and physiological characteristics of the recurrent parent are recovered in the converted plant, in addition to the single gene or a limited number of genes transferred from the donor parent.

A “line”, as used herein, refers to a population of plants derived from a single cross, backcross or selfing. The individual offspring plants are not necessarily identical to one another. It is possible that individual offspring plants are not vigorous, fertile or self-compatible due to natural variability. However, plants that are vigorous, fertile and self-compatible can be easily identified in a line and used for additional breeding purpose.

The selection of a suitable recurrent parent is an important step for a successful backcrossing procedure. The goal of a backcross protocol is to alter or substitute a single trait or characteristic in the original variety. To accomplish this, a single gene of the recurrent variety is modified, substituted or supplemented with the desired gene from the donor parent, while retaining essentially all of the rest of the desired genes, and therefore the desired physiological and morphological constitution of the original variety. The choice of the particular donor parent will depend on the purpose of the backcross. One of the major purposes is to add some commercially desirable, agronomically important traits to the recurrent parent (e.g. expression of a terpene synthase gene or a transporter gene). The exact backcrossing protocol will depend on the characteristic or trait being altered or added (e.g. expression of a terpene synthase gene or a transporter gene) to determine an appropriate testing protocol. Although backcrossing methods are simplified when the characteristic being transferred is a dominant allele, a recessive allele may also be transferred. In this instance, it may be necessary to introduce a test of the progeny to determine if the desired characteristic (e.g. expression of a terpene synthase gene or a transporter gene) has been successfully transferred. Preferably, such genes are monitored by molecular markers, as discussed below.

According to one embodiment, the introgression comprises allelic variation(s) in compared to a genome of a recurrent parent of the plant.

As used herein, the term “allelic variation” refers to the presence or number of different allele forms at a particular gene locus.

According to one embodiment, the allelic variation is in a region spanning no more than 1,000,000 base pairs.

According to one embodiment, the allelic variation is in a region spanning no more than 500,000 base pairs.

According to one embodiment, the allelic variation is in a region spanning no more than 250,000 base pairs.

According to one embodiment, the allelic variation is in a region spanning no more than 100,000 base pairs.

According to one embodiment, the allelic variation is in a region spanning no more than 50,000 base pairs.

According to one embodiment, the allelic variation is in a region spanning no more than 40,000 base pairs.

According to one embodiment, the allelic variation is in a region spanning no more than 30,000 base pairs.

According to one embodiment, the allelic variation is in a region spanning no more than 20,000 base pairs.

According to one embodiment, the allelic variation is in a region spanning no more than 10,000 base pairs.

According to one embodiment, the allelic variation is in a region spanning no more than 7500 base pairs.

According to one embodiment, the allelic variation is in a region spanning no more than 5000 base pairs.

According to one embodiment, the allelic variation is in a region spanning no more than 4000 base pairs.

According to one embodiment, the allelic variation is in a region spanning no more than 3000 base pairs.

According to one embodiment, the allelic variation is in a region spanning no more than 2000 base pairs.

According to one embodiment, the allelic variation is in a region spanning no more than 1000 base pairs.

According to one embodiment, the allelic variation is in a region spanning no more than 750 base pairs.

According to one embodiment, the allelic variation is in a region spanning no more than 500 base pairs.

According to one embodiment, the allelic variation comprises a polynucleotide sequence encoding a polypeptide comprising an amino acid sequence as set forth in any one of SEQ ID NO: 1-45, or an amino acid sequence which is at least 80%, at least 85%; at least 90%, at least 91%, at least 92%, at least 93%, at least 94%, at least 95%, at least 96%, at least 97%, at least 98%, at least 99% or 100% identical to SEQ ID NO: 1-45 and provided it is not a sequence which is that of the recurrent patent and having a terpene synthase activity.

According to a specific embodiment, the allelic variation comprises a polynucleotide sequence encoding a polypeptide comprising an amino acid sequence which is 98% identical to SEQ ID NO: 1-45 and provided it is not a sequence which is that of the recurrent patent and having a terpene synthase activity.

According to a specific embodiment, the allelic variation comprises a polynucleotide sequence encoding a polypeptide comprising an amino acid sequence which is 99% identical to SEQ ID NO: 1-45 and provided it is not a sequence which is that of the recurrent patent and having a terpene synthase activity.

According to a specific embodiment, the allelic variation comprises a polynucleotide sequence encoding a polypeptide comprising an amino acid sequence which is 100% identical to SEQ ID NO: 1-45 and provided it is not a sequence which is that of the recurrent patent and having a terpene synthase activity.

According to one embodiment, the allelic variation comprises a polynucleotide sequence comprising a nucleic acid sequence as set forth in any one of SEQ ID NO: 58-102, or a nucleic acid sequence which is at least 80%, at least 85%; at least 90%, at least 91%, at least 92%, at least 93%, at least 94%, at least 95%, at least 96%, at least 97%, at least 98%, at least 99% or 100% identical to SEQ ID NO: 58-102 and provided it is not a sequence which is that of the recurrent patent and having a terpene synthase activity.

According to one embodiment, the allelic variation comprises a polynucleotide sequence encoding a polypeptide comprising an amino acid sequence as set forth in any one of SEQ ID NO: 46-57, 116, 118, 120, 122, 124, 126, 128 and 130 or an amino acid sequence which is at least 80%, at least 85%; at least 90%, at least 91%, at least 92%, at least 93%, at least 94%, at least 95%, at least 96%, at least 97%, at least 98%, at least 99% or 100% identical to SEQ ID NO: 46-57, 116, 118, 120, 122, 124, 126, 128 and 130, and provided it is not a sequence which is that of the recurrent patent and having a transporter activity.

According to one embodiment, the allelic variation comprises a polynucleotide sequence encoding a polypeptide comprising an amino acid sequence which is 95% identical to SEQ ID NO: 46-57, 116, 118, 120, 122, 124, 126, 128 and 130, and provided it is not a sequence which is that of the recurrent patent and having a transporter activity.

According to one embodiment, the allelic variation comprises a polynucleotide sequence encoding a polypeptide comprising an amino acid sequence which is 97% identical to SEQ ID NO: 46-57, 116, 118, 120, 122, 124, 126, 128 and 130, and provided it is not a sequence which is that of the recurrent patent and having a transporter activity.

According to one embodiment, the allelic variation comprises a polynucleotide sequence encoding a polypeptide comprising an amino acid sequence which is 99% identical to SEQ ID NO: 46-57, 116, 118, 120, 122, 124, 126, 128 and 130, and provided it is not a sequence which is that of the recurrent patent and having a transporter activity.

According to one embodiment, the allelic variation comprises a polynucleotide sequence encoding a polypeptide comprising an amino acid sequence which is 100% identical to SEQ ID NO: 46-57, 116, 118, 120, 122, 124, 126, 128 and 130, and provided it is not a sequence which is that of the recurrent patent and having a transporter activity.

According to one embodiment, the allelic variation comprises a polynucleotide sequence comprising a nucleic acid sequence as set forth in any one of SEQ ID NO: 103-115, 117, 119, 121, 123, 125, 127 and 129, or a nucleic acid sequence which is at least 80%, at least 85%; at least 90%, at least 91%, at least 92%, at least 93%, at least 94%, at least 95%, at least 96%, at least 97%, at least 98%, at least 99% or 100% identical to SEQ ID NO: 103-115, 117, 119, 121, 123, 125, 127 and 129, and provided it is not a sequence which is that of the recurrent patent and having a transporter activity.

According to a specific embodiment, the allelic variation comprises a polynucleotide sequence encoding a polypeptide comprising an amino acid sequence as set forth in any one of SEQ ID NO: 1-57, 116, 118, 120, 122, 124, 126, 128 and 130.

According to a specific embodiment, the allelic variation comprises a polynucleotide sequence as set forth in any one of SEQ ID NO: 58-115, 117, 119, 121, 123, 125, 127 and 129.

According to one embodiment, the allelic variation comprises a polynucleotide sequence encoding a polypeptide having a terpene synthase activity (as discussed above) and a polypeptide having a transporter activity (as discussed above).

According to one embodiment, a plant co-expressing a polypeptide having a terpene synthase activity and a polypeptide having a transporter activity can be obtained by crossing, e.g. taking a plant which has been bred to express (or comprise the introgression) the TPS gene and crossing with a plant that has been bred to express (or comprise the introgression) the transporter gene as described herein.

According to one embodiment, a plant co-expressing polypeptides having two separate terpene synthase activities can be obtained by crossing, e.g. taking a plant which has been bred to express (or comprise the introgression) the first TPS gene and crossing with a plant that has been bred to express (or comprise the introgression) the second TPS gene as described herein.

According to one embodiment, a plant co-expressing polypeptides having two separate transporter activities can be obtained by crossing, e.g. taking a plant which has been bred to express (or comprise the introgression) the first transported gene and crossing with a plant that has been bred to express (or comprise the introgression) the second transporter gene as described herein.

Accordingly, plants may be obtained co-expressing additional polypeptides having a terpene synthase activity and a polypeptide having a transporter activity. Such a determination can be carried out by a person of skill in the art.

According to another aspect of the invention there is provided an isolated polypeptide comprising an amino acid sequence at least 80%, at least 85%; at least 90%, at least 91%, at least 92%, at least 93%, at least 94%, at least 95%, at least 96%, at least 97%, at least 98%, at least 99% or 100% identical to the amino acid sequence selected from the group consisting of SEQ ID NOs: 1-45, wherein the polypeptide, when expressed in an organism (e.g. non-human organism, e.g. plant or part thereof), is capable of modulating the synthesis of a terpene of interest.

According to a specific embodiment, the isolated polypeptide comprises an amino acid sequence 98% identical to the amino acid sequence selected from the group consisting of SEQ ID NOs: 1-45, wherein the polypeptide, when expressed in an organism (e.g. non-human organism, e.g. plant or part thereof), is capable of modulating the synthesis of a terpene of interest.

According to a specific embodiment, the isolated polypeptide comprises an amino acid sequence 99% identical to the amino acid sequence selected from the group consisting of SEQ ID NOs: 1-45, wherein the polypeptide, when expressed in an organism (e.g. non-human organism, e.g. plant or part thereof), is capable of modulating the synthesis of a terpene of interest.

According to a specific embodiment, the isolated polypeptide comprises an amino acid sequence 100% identical to the amino acid sequence selected from the group consisting of SEQ ID NOs: 1-45, wherein the polypeptide, when expressed in an organism (e.g. non-human organism, e.g. plant or part thereof), is capable of modulating the synthesis of a terpene of interest.

According to another aspect of the invention there is provided an isolated polypeptide comprising an amino acid sequence at least 80%, at least 85%; at least 90%, at least 91%, at least 92%, at least 93%, at least 94%, at least 95%, at least 96%, at least 97%, at least 98%, at least 99% or 100% identical to the amino acid sequence selected from the group consisting of SEQ ID NOs: 46-57, 116, 118, 120, 122, 124, 126, 128 and 130, wherein the polypeptide, when expressed in an organism (e.g. non-human organism, e.g. plant or part thereof), is capable of modulating transport of metabolites.

According to a specific embodiment, the isolated polypeptide comprises an amino acid sequence 95% identical to the amino acid sequence selected from the group consisting of SEQ ID NOs: 46-57, 116, 118, 120, 122, 124, 126, 128 and 130, wherein the polypeptide, when expressed in an organism (e.g. non-human organism, e.g. plant or part thereof), is capable of modulating transport of metabolites.

According to a specific embodiment, the isolated polypeptide comprises an amino acid sequence 97% identical to the amino acid sequence selected from the group consisting of SEQ ID NOs: 46-57, 116, 118, 120, 122, 124, 126, 128 and 130, wherein the polypeptide, when expressed in an organism (e.g. non-human organism, e.g. plant or part thereof), is capable of modulating transport of metabolites.

According to a specific embodiment, the isolated polypeptide comprises an amino acid sequence 99% identical to the amino acid sequence selected from the group consisting of SEQ ID NOs: 46-57, 116, 118, 120, 122, 124, 126, 128 and 130, wherein the polypeptide, when expressed in an organism (e.g. non-human organism, e.g. plant or part thereof), is capable of modulating transport of metabolites.

According to a specific embodiment, the isolated polypeptide comprises an amino acid sequence 100% identical to the amino acid sequence selected from the group consisting of SEQ ID NOs: 46-57, 116, 118, 120, 122, 124, 126, 128 and 130, wherein the polypeptide, when expressed in an organism (e.g. non-human organism, e.g. plant or part thereof), is capable of modulating transport of metabolites.

The term “isolated” refers to at least partially separated from the natural environment e.g., from an organism e.g. from a whole plant.

As used herein, the terms “polypeptide” or “heterologous polypeptide” refer to a polypeptide produced by recombinant DNA techniques, i.e., produced from cells transformed by an exogenous nucleic acid (e.g. nucleic acid construct) encoding the polypeptide. The polypeptide can be foreign to the cell or a homologous polypeptide derived from a nucleic acid sequence not from its natural location and expression level in the genome of the cell.

Homology (e.g., percent homology, sequence identity+sequence similarity) can be determined using any homology comparison software computing a pairwise sequence alignment.

As used herein, “sequence identity” or “identity” in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff JG. [Amino acid substitution matrices from protein blocks. Proc. Natl. Acad. Sci. U.S.A. 1992, 89(22): 10915-9].

Identity (e.g., percent homology) can be determined using any homology comparison software, including for example, the BlastN software of the National Center of Biotechnology Information (NCBI) such as by using default parameters.

According to some embodiments of the invention, the identity is a global identity, i.e., an identity over the entire amino acid or nucleic acid sequences of the invention and not over portions thereof.

According to some embodiments of the invention, the term “homology” or “homologous” refers to identity of two or more nucleic acid sequences; or identity of two or more amino acid sequences; or the identity of an amino acid sequence to one or more nucleic acid sequence.

According to some embodiments of the invention, the homology is a global homology, i.e., a homology over the entire amino acid or nucleic acid sequences of the invention and not over portions thereof.

The degree of homology or identity between two or more sequences can be determined using various known sequence comparison tools. Following is a non-limiting description of such tools which can be used along with some embodiments of the invention.

When starting with a polynucleotide sequence and comparing to other polynucleotide sequences the EMBOSS-6.0.1 Needleman-Wunsch algorithm (available from emboss(dot)sourceforge(dot)net/apps/cvs/emboss/apps/needle(dot)html) can be used with the following default parameters: (EMBOSS-6.0.1) gapopen=10; gapextend=0.5: datafile=EDNAFULL; brief=YES.

According to some embodiments of the invention, the parameters used with the EMBOSS-6.0.1 Needleman-Wunsch algorithm are gapopen=10; gapextend=0.2; datafile=EDNAFULL; brief=YES.

According to some embodiments of the invention, the threshold used to determine homology using the EMBOSS-6.0.1 Needleman-Wunsch algorithm for comparison of polynucleotides with polynucleotides is 80%, 81%, 82%, 83%, 84%, 85%, 86%, 87%, 88%, 89%, 90%, 91%, 92%, 93%, 94%, 95%, 96%, 97%, 98%, 99%, or 100%.

According to some embodiment, determination of the degree of homology further requires employing the Smith-Waterman algorithm (for protein-protein comparison or nucleotide-nucleotide comparison).

Default parameters for GenCore 6.0 Smith-Waterman algorithm include: model=sw.model.

According to some embodiments of the invention, the threshold used to determine homology using the Smith-Waterman algorithm is 80%, 81%, 82%, 83%, 84%, 85%, 86%, 87%, 88%, 89%, 90%, 91%, 92%, 93%, 94%, 95%, 96%, 97%, 98%, 99%, or 100%.

According to some embodiments of the invention, the global homology is performed on sequences which are pre-selected by local homology to the polypeptide or polynucleotide of interest (e.g., 60% identity over 60% of the sequence length), prior to performing the global homology to the polypeptide or polynucleotide of interest (e.g., 80% global homology on the entire sequence). For example, homologous sequences are selected using the BLAST software with the Blastp and tBlastn algorithms as filters for the first stage, and the needle (EMBOSS package) or Frame+ algorithm alignment for the second stage. Local identity (Blast alignments) is defined with a very permissive cutoff −60% Identity on a span of 60% of the sequences lengths because it is used only as a filter for the global alignment stage. In this specific embodiment (when the local identity is used), the default filtering of the Blast package is not utilized (by setting the parameter “−FF”).

In the second stage, homologs are defined based on a global identity of at least 80% to the core gene polypeptide sequence. According to some embodiments the homology is a local homology or a local identity.

Local alignments tools include, but are not limited to the BlastP, BlastN, BlastX or TBLASTN software of the National Center of Biotechnology Information (NCBI), FASTA, and the Smith-Waterman algorithm.

According to a specific embodiment, homology is determined using BlastN version 2.7.1+ with the following default parameters: task=blastn, evalue=10, strand=both, gap opening penalty=5, gap extension penalty=2, match=1, mismatch=−1, word size=11, max scores=25, max alignments=15, query filter=dust, query gentetic code—n/a, matrix=no default.

According to one embodiment, there is provided an isolated polynucleotide encoding the polypeptide of some embodiments of the invention.

According to one embodiment, the polynucleotide comprises the nucleic acid sequence as set forth in SEQ ID NO: 58-115, 117, 119, 121, 123, 125, 127 and 129.

According to another aspect of the invention there is provided an organism comprising a genome having been genetically modified to express a polypeptide having a terpene synthase activity, the polypeptide being at least 80%, at least 85%; at least 90%, at least 91%, at least 92%, at least 93%, at least 94%, at least 95%, at least 96%, at least 97%, at least 98%, at least 99% or 100% identical to an amino acid sequence selected from the group consisting of SEQ ID NO: 1-45.

According to a specific embodiment there is provided an organism comprising a genome having been genetically modified to express a polypeptide having a terpene synthase activity, the polypeptide being 98%, 99% or 100% identical to an amino acid sequence selected from the group consisting of SEQ ID NO: 1-45.

According to another aspect of the invention there is provided a cell comprising a genome having been genetically modified to express a polypeptide having a terpene synthase activity, the polypeptide being at least 80%, at least 85%; at least 90%, at least 91%, at least 92%, at least 93%, at least 94%, at least 95%, at least 96%, at least 97%, at least 98%, at least 99% or 100% identical to an amino acid sequence selected from the group consisting of SEQ ID NO: 1-45.

According to a specific embodiment there is provided a cell comprising a genome having been genetically modified to express a polypeptide having a terpene synthase activity, the polypeptide being 98%, 99% or 100% identical to an amino acid sequence selected from the group consisting of SEQ ID NO: 1-45.

According to another aspect of the invention there is provided a plant comprising a genome having been genetically modified to express a polypeptide having a terpene synthase activity, the polypeptide being at least 80%, at least 85%; at least 90%, at least 91%, at least 92%, at least 93%, at least 94%, at least 95%, at least 96%, at least 97%, at least 98%, at least 99% or 100% identical to an amino acid sequence selected from the group consisting of SEQ ID NO: 1-45.

According to a specific embodiment there is provided a plant comprising a genome having been genetically modified to express a polypeptide having a terpene synthase activity, the polypeptide being 98%, 99% or 100% identical to an amino acid sequence selected from the group consisting of SEQ ID NO: 1-45.

According to another aspect of the invention there is provided a method of modulating terpene synthesis in an organism, the method comprising over-expressing within at least one cell of the organism a polypeptide having a terpene synthase activity, the polypeptide being at least 80%, at least 85%; at least 90%, at least 91%, at least 92%, at least 93%, at least 94%, at least 95%, at least 96%, at least 97%, at least 98%, at least 99% or 100% identical to an amino acid sequence selected from the group consisting of SEQ ID NO: 1-45, thereby modulating terpene synthesis in the organism.

According to a specific embodiment there is provided a method of modulating terpene synthesis in an organism, the method comprising over-expressing within at least one cell of the organism a polypeptide having a terpene synthase activity, the polypeptide being 98%, 99% or 100% identical to an amino acid sequence selected from the group consisting of SEQ ID NO: 1-45, thereby modulating terpene synthesis in the organism.

According to another aspect of the invention there is provided a method of modulating terpene synthesis in a cell of interest, the method comprising over-expressing within at least one cell of interest a polypeptide having a terpene synthase activity, the polypeptide being at least 80%, at least 85%; at least 90%, at least 91%, at least 92%, at least 93%, at least 94%, at least 95%, at least 96%, at least 97%, at least 98%, at least 99% or 100% identical to an amino acid sequence selected from the group consisting of SEQ ID NO: 1-45, thereby modulating terpene synthesis in the cell of interest.

According to a specific embodiment there is provided a method of modulating terpene synthesis in a cell of interest, the method comprising over-expressing within at least one cell of interest a polypeptide having a terpene synthase activity, the polypeptide being 98%, 99% or 100% identical to an amino acid sequence selected from the group consisting of SEQ ID NO: 1-45, thereby modulating terpene synthesis in the cell of interest.

According to another aspect of the invention there is provided a method of modulating terpene synthesis in a plant, the method comprising over-expressing within the plant or part thereof a polypeptide having a terpene synthase activity, the polypeptide being at least 80%, at least 85%; at least 90%, at least 91%, at least 92%, at least 93%, at least 94%, at least 95%, at least 96%, at least 97%, at least 98%, at least 99% or 100% identical to an amino acid sequence selected from the group consisting of SEQ ID NO: 1-45, thereby modulating terpene synthesis in the plant.

According to a specific embodiment there is provided a method of modulating terpene synthesis in a plant, the method comprising over-expressing within the plant or part thereof a polypeptide having a terpene synthase activity, the polypeptide being 98%, 99% or 100% identical to an amino acid sequence selected from the group consisting of SEQ ID NO: 1-45, thereby modulating terpene synthesis in the plant.

According to a specific embodiment, the amino acid sequence is at least 85% identical to SEQ ID NO: 1-45.

According to a specific embodiment, the amino acid sequence is at least 90% identical to SEQ ID NO: 1-45.

According to a specific embodiment, the amino acid sequence is at least 95% identical to SEQ ID NO: 1-45.

According to a specific embodiment, the amino acid sequence is at least 97% identical to SEQ ID NO: 1-45.

According to a specific embodiment, the amino acid sequence is at least 98% identical to SEQ ID NO: 1-45.

According to a specific embodiment, the amino acid sequence is at least 99% identical to SEQ ID NO: 1-45.

According to a specific embodiment, the amino acid sequence is as set forth in SEQ ID NO: 1-45.

According to a specific embodiment, the amino acid sequence has a terpene synthase activity.

According to one embodiment, the polypeptide sequence is encoded by a nucleic acid sequence as set forth in any one of SEQ ID NO: 58-102 or a nucleic acid sequence which is at least 80%, at least 85%; at least 90%, at least 91%, at least 92%, at least 93%, at least 94%, at least 95%, at least 96%, at least 97%, at least 98%, at least 99% or 100% identical to SEQ ID NO: 58-102.

According to another aspect of the invention there is provided an organism comprising a genome having been genetically modified to express a polypeptide having a transporter activity, the polypeptide being at least 80%, at least 85%; at least 90%, at least 91%, at least 92%, at least 93%, at least 94%, at least 95%, at least 96%, at least 97%, at least 98%, at least 99% or 100% identical to an amino acid sequence selected from the group consisting of SEQ ID NO: 46-57, 116, 118, 120, 122, 124, 126, 128 and 130.

According to a specific embodiment there is provided an organism comprising a genome having been genetically modified to express a polypeptide having a transporter activity, the polypeptide being 95%, 96%, 97%, 98%, 99% or 100% identical to an amino acid sequence selected from the group consisting of SEQ ID NO: 46-57, 116, 118, 120, 122, 124, 126, 128 and 130.

According to another aspect of the invention there is provided a cell comprising a genome having been genetically modified to express a polypeptide having a transporter activity, the polypeptide being at least 80%, at least 85%; at least 90%, at least 91%, at least 92%, at least 93%, at least 94%, at least 95%, at least 96%, at least 97%, at least 98%, at least 99% or 100% identical to an amino acid sequence selected from the group consisting of SEQ ID NO: 46-57, 116, 118, 120, 122, 124, 126, 128 and 130.

According to a specific embodiment there is provided a cell comprising a genome having been genetically modified to express a polypeptide having a transporter activity, the polypeptide being 95%, 96%, 97%, 98%, 99% or 100% identical to an amino acid sequence selected from the group consisting of SEQ ID NO: 46-57, 116, 118, 120, 122, 124, 126, 128 and 130.

According to another aspect of the invention there is provided a plant comprising a genome having been genetically modified to express a polypeptide having a transporter activity, the polypeptide being at least 80%, at least 85%; at least 90%, at least 91%, at least 92%, at least 93%, at least 94%, at least 95%, at least 96%, at least 97%, at least 98%, at least 99% or 100% identical to an amino acid sequence selected from the group consisting of SEQ ID NO: 46-57, 116, 118, 120, 122, 124, 126, 128 and 130.

According to a specific embodiment there is provided a plant comprising a genome having been genetically modified to express a polypeptide having a transporter activity, the polypeptide being 95%, 96%, 97%, 98%, 99% or 100% identical to an amino acid sequence selected from the group consisting of SEQ ID NO: 46-57, 116, 118, 120, 122, 124, 126, 128 and 130.

According to another aspect of the invention there is provided a method of modulating transport of metabolites in an organism, the method comprising over-expressing within at least one cell of the organism a polypeptide having a transporter activity, the polypeptide being at least 80%, at least 85%; at least 90%, at least 91%, at least 92%, at least 93%, at least 94%, at least 95%, at least 96%, at least 97%, at least 98%, at least 99% or 100% identical to an amino acid sequence selected from the group consisting of SEQ ID NO: 46-57, 116, 118, 120, 122, 124, 126, 128 and 130, thereby modulating the transport of the metabolites in the organism.

According to a specific embodiment there is provided a method of modulating transport of metabolites in an organism, the method comprising over-expressing within at least one cell of the organism a polypeptide having a transporter activity, the polypeptide being 95%, 96%, 97%, 98%, 99% or 100% identical to an amino acid sequence selected from the group consisting of SEQ ID NO: 46-57, 116, 118, 120, 122, 124, 126, 128 and 130, thereby modulating the transport of the metabolites in the organism.

According to another aspect of the invention there is provided a method of modulating transport of metabolites in a cell of interest, the method comprising over-expressing within at least one cell of the organism a polypeptide having a transporter activity, the polypeptide being at least 80%, at least 85%; at least 90%, at least 91%, at least 92%, at least 93%, at least 94%, at least 95%, at least 96%, at least 97%, at least 98%, at least 99% or 100% identical to an amino acid sequence selected from the group consisting of SEQ ID NO: 46-57, 116, 118, 120, 122, 124, 126, 128 and 130, thereby modulating transport of metabolites in the cell of interest.

According to a specific embodiment there is provided a method of modulating transport of metabolites in a cell of interest, the method comprising over-expressing within at least one cell of the organism a polypeptide having a transporter activity, the polypeptide being 95%, 96%, 97%, 98%, 99% or 100% identical to an amino acid sequence selected from the group consisting of SEQ ID NO: 46-57, 116, 118, 120, 122, 124, 126, 128 and 130, thereby modulating transport of metabolites in the cell of interest.

According to another aspect of the invention there is provided a method of modulating transport of metabolites in a plant, the method comprising over-expressing within the plant or part thereof a polypeptide having a transporter activity, the polypeptide being at least 80%, at least 85%; at least 90%, at least 91%, at least 92%, at least 93%, at least 94%, at least 95%, at least 96%, at least 97%, at least 98%, at least 99% or 100% identical to an amino acid sequence selected from the group consisting of SEQ ID NO: 46-57, 116, 118, 120, 122, 124, 126, 128 and 130, thereby modulating the transport of the metabolites in the plant.

According to a specific embodiment there is provided a method of modulating transport of metabolites in a plant, the method comprising over-expressing within the plant or part thereof a polypeptide having a transporter activity, the polypeptide being 95%, 96%, 97%, 98%, 99% or 100% identical to an amino acid sequence selected from the group consisting of SEQ ID NO: 46-57, 116, 118, 120, 122, 124, 126, 128 and 130, thereby modulating the transport of the metabolites in the plant.

According to a specific embodiment, the amino acid sequence is at least 85% identical to SEQ ID NO: 46-57, 116, 118, 120, 122, 124, 126, 128 and 130.

According to a specific embodiment, the amino acid sequence is at least 90% identical to SEQ ID NO: 46-57, 116, 118, 120, 122, 124, 126, 128 and 130.

According to a specific embodiment, the amino acid sequence is at least 95% identical to SEQ ID NO: 46-57, 116, 118, 120, 122, 124, 126, 128 and 130.

According to a specific embodiment, the amino acid sequence is at least 96% identical to SEQ ID NO: 46-57, 116, 118, 120, 122, 124, 126, 128 and 130.

According to a specific embodiment, the amino acid sequence is at least 97% identical to SEQ ID NO: 46-57, 116, 118, 120, 122, 124, 126, 128 and 130.

According to a specific embodiment, the amino acid sequence is at least 98% identical to SEQ ID NO: 46-57, 116, 118, 120, 122, 124, 126, 128 and 130.

According to a specific embodiment, the amino acid sequence is at least 99% identical to SEQ ID NO: 46-57, 116, 118, 120, 122, 124, 126, 128 and 130.

According to a specific embodiment, the amino acid sequence is as set forth in SEQ ID NO: 46-57, 116, 118, 120, 122, 124, 126, 128 and 130.

According to a specific embodiment, the amino acid sequence has a transporter activity.

According to one embodiment, the polypeptide sequence is encoded by a nucleic acid sequence as set forth in any one of SEQ ID NO: 103-115, 117, 119, 121, 123, 125, 127 and 129, or a nucleic acid sequence which is at least 80%, at least 85%; at least 90%, at least 91%, at least 92%, at least 93%, at least 94%, at least 95%, at least 96%, at least 97%, at least 98%, at least 99% or 100% identical to SEQ ID NO: 103-115, 117, 119, 121, 123, 125, 127 and 129.

As used herein, the term “genetically modified” refers to an organism e.g. non-human organism, e.g. plant (e.g. cannabis plant) comprising an exogenous nucleic acid sequence. The organism (e.g. plant) may be transgenic or non-transgenic (i.e., wherein a cell of the organism, e.g. plant, does not comprise foreign regulatory elements such as viral components, e.g., exogenous promoter).

The term “over-expressing” refers to an expression of a polypeptide at least about 10%, at least about 20%, at least about 30%, at least about 40%, at least about 50%, at least about 60%, at least about 70%, at least about 80%, at least about 90%, at least about 95% or about 100% higher as compared to a non-modified organism e.g. plant (e.g. recurrent plant).

According to one embodiment, modulating comprises enhancing the expression of the terpene of interest by at least about 10%, at least about 20%, at least about 30%, at least about 40%, at least about 50%, at least about 60%, at least about 70%, at least about 80%, at least about 90%, at least about 95% or about 100% as compared to its expression in a non-genetically modified organism e.g. plant (e.g. recurrent plant).

According to one embodiment, modulating comprises enhancing the transport of metabolites by at least about 10%, at least about 20%, at least about 30%, at least about 40%, at least about 50%, at least about 60%, at least about 70%, at least about 80%, at least about 90%, at least about 95% or about 100% as compared to transport of metabolites in a non-genetically modified organism e.g. plant (e.g. recurrent plant).

Such enhanced transport enables less self-toxicity in a cell e.g. plant cell, which ultimately leads to higher production of metabolites (e.g. secondary metabolites including terpenes and cannabinoids in plant cells). Such metabolites may be recovered from the organism e.g. plant as described hereinbelow.

According to one embodiment of the invention, the method comprises co-expressing within the organism (e.g. non-human organism, e.g. plant or part thereof, bacteria, yeast, insect, etc.) a polypeptide having a terpene synthase activity and a polypeptide having a transporter activity. Such co-expression can be effected concomitantly or at separate times (e.g. within minutes, hours, days, weeks or months of each other).

According to one embodiment, the method comprises co-expressing within the organism (e.g. non-human organism, e.g. plant or part thereof, bacteria, yeast, insect, etc.) polypeptides having two separate terpene synthase activities. Such co-expression can be effected concomitantly or at separate times (e.g. within minutes, hours, days, weeks or months of each other).

According to one embodiment, the method comprises co-expressing within the organism (e.g. non-human organism, e.g. plant or part thereof, bacteria, yeast, insect, etc.) polypeptides having two separate transporter activities. Such co-expression can be effected concomitantly or at separate times (e.g. within minutes, hours, days, weeks or months of each other).

According to one embodiment, the method comprises introducing into at least one cell of the organism (e.g. non-human organism, e.g. plant or part thereof, bacteria, yeast, insect, etc.) an exogenous polynucleotide encoding the polypeptide.

Transgenes can be introduced into the organism (e.g. non-human organism, e.g. plant, bacteria, yeast, insect, etc.) using any of an assortment of established recombinant methods well-known to persons skilled in the art.

According to one embodiment, methods of genetic transformation are utilized wherein the gene is isolated from the chromosome of a donor variety (e.g. donor plant variety), or wherein a synthetic gene is produced, and wherein the isolated or synthetic gene is then introduced into the recipient (e.g. recipient plant) by genetic transformation techniques.

According to one embodiment, a nucleic acid (e.g. DNA) sequence comprising one or more of the genes as defined herein and in Tables 1 and 7 below may be used for the production of an organism (e.g. non-human organism, e.g. plant, bacteria, yeast, insect, etc.) having an additional agronomically desirable trait (e.g. expression of a terpene synthase gene or a transporter gene).

For transgenic methods of transfer a nucleic acid sequence comprising a desirable gene for an agronomically desirable trait (e.g. expression of a terpene synthase gene or a transporter gene) may be isolated from a donor organism e.g. plant by using methods known in the art (or may be synthetically produced) and the isolated nucleic acid sequence may be transferred to the recipient organism e.g. plant by transgenic methods for transformation (e.g. plant transformation), for instance by means of a vector, in a gamete, or in any other suitable transfer element, such as a bombardment with a particle coated with the nucleic acid sequence, as further discussed below. Plant transformation generally involves the construction of a vector with an expression cassette that will function in plant cells.

According to one embodiment, introducing the exogenous polynucleotide into the at least one cell comprises transforming the polynucleotide or a construct comprising same into the at least one cell.

Constructs useful in the methods according to some embodiments of the invention may be constructed using recombinant DNA technology well known to persons skilled in the art. The gene constructs may be inserted into vectors, which may be commercially available, suitable for transforming into plants and suitable for expression of the gene of interest in the transformed cells. The genetic construct can be an expression vector wherein the nucleic acid sequence is operably linked to one or more regulatory sequences allowing expression in the plant cells.

According to one embodiment, the regulatory sequence is a cis-acting regulatory element for directing expression of the nucleic acid sequence in a cell e.g. plant cell.

In a particular embodiment of some embodiments of the invention the regulatory sequence (e.g. sequence of the cis-acting regulatory element) is a plant-expressible promoter.

As used herein the phrase “plant-expressible” refers to a promoter sequence, including any additional regulatory elements added thereto or contained therein, is at least capable of inducing, conferring, activating or enhancing expression in a plant cell, tissue or organ, preferably a monocotyledonous or dicotyledonous plant cell, tissue, or organ. Suitable promoters which may be used in accordance with the present teachings include constitutive promoters, seed preferred promoters, flower specific promoters, as discussed in U.S. Patent Application Nos. 20180371484 and 20160348125, incorporated herein by reference in their entirety.

According to a specific embodiment, the promoter is a trichome-specific promoter, see a labdane diterpene Z-abienol in tobacco glandular trichomes (Sallaud C. el al., Plant J. (2013) 72:1-17), a cembratrien-ol synthase gene in Nicotiana sylvestris (Ennajdaoui H. et al., Plant Mol. Biol. (2010) 73: 673-685) and a BAHD acetyltransferase in tomato trichomes. (Schilmiller A. L. et al. Proc. Natl. Acad. Si. USA. (2012) 109:16377-16382).

Nucleic acid sequences of the polypeptides of some embodiments of the invention may be optimized for expression in a specific organism e.g. plant expression. Examples of such sequence modifications include, but are not limited to, an altered G/C content to more closely approach that typically found in the plant species of interest, and the removal of codons atypically found in the plant species commonly referred to as codon optimization. Codon optimization tables are provided on-line e.g. at the Codon Usage Database through the NIAS (National Institute of Agrobiological Sciences) DNA bank in Japan (www(dot)kazusa(dot)or(dot)jp/codon/). The Codon Usage Database contains codon usage tables for a number of different species, with each codon usage table having been statistically determined based on the data present in Genbank.

Thus, some embodiments of the invention encompasses nucleic acid sequences described hereinabove; fragments thereof, sequences hybridizable therewith, sequences homologous thereto, sequences orthologous thereto, sequences encoding similar polypeptides with different codon usage, altered sequences characterized by mutations, such as deletion, insertion or substitution of one or more nucleotides, either naturally occurring or man induced, either randomly or in a targeted fashion.

Cells, e.g. plant cells, may be transformed stably or transiently with the nucleic acid constructs of some embodiments of the invention. In stable transformation, the nucleic acid molecule of some embodiments of the invention is integrated into the genome, e.g. plant genome, and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276) including Agrobacterium-mediated gene transfer and direct DNA uptake (e.g. electroporation, microinjection, microparticle bombardment) as discussed in U.S. Patent Application Nos. 20180371484 and 20160348125, both incorporated herein by reference in their entirety.

Although stable transformation is presently preferred, transient transformation of cells e.g. leaf cells, meristematic cells or the whole plant, is also envisaged by some embodiments of the invention.

Transient transformation can be effected by any of the direct DNA transfer methods described above or by viral infection using modified viruses (e.g. plant viruses).

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology. Viral Vectors, Cold Spring Harbor Laboratory, New York, pp. 172-189 (1988). Pseudovirus particles for use in expressing foreign DNA in many hosts, including plants, is described in WO 87/06261.

Construction of RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

In addition to the above, the nucleic acid molecule of some embodiments of the invention can also be introduced into a chloroplast genome thereby enabling chloroplast expression.

A technique for introducing exogenous nucleic acid sequences to the genome of the chloroplasts is known. Further details relating to this technique are found in U.S. Pat. Nos. 4,945,050; and 5,693,507 which are incorporated herein by reference.

According to one embodiment, the expression vectors can include at least one marker gene that allows transformed cells containing the marker to be either recovered by negative selection (by inhibiting the growth of cells that do not contain the selectable marker gene), or by positive selection (by screening for the product encoded by the marker gene). Many commonly used selectable marker genes for transformation are known in the art, and include, for example, genes that code for enzymes that metabolically detoxify a selective chemical agent which may be an antibiotic or a herbicide, or genes that encode an altered target which is insensitive to an inhibitor.

According to one embodiment, the marker is a toxic selection marker. An exemplary toxic selection marker that can be used as a marker is, without being limited to, allyl alcohol selection using the Alcohol dehydrogenase (ADH1) gene. ADH1, comprising a group of dehydrogenase enzymes which catalyse the interconversion between alcohols and aldehydes or ketones with the concomitant reduction of NAD+ or NADP+, breaks down alcoholic toxic substances within tissues. For example, plants harboring reduced ADH1 expression exhibit increase tolerance to allyl alcohol. Accordingly, plants with reduced ADH1 are resistant to the toxic effect of allyl alcohol.

Additionally or alternatively, a fluorescent protein can be used which emits fluorescence and is typically detectable by flow cytometry, microscopy or any fluorescent imaging system, therefore can be used as a basis for selection of cells expressing such a protein.

Examples of fluorescent proteins that can be used as markers are, without being limited to, the Green Fluorescent Protein (GFP), the Blue Fluorescent Protein (BFP) and the red fluorescent proteins (e.g. dsRed, mCherry, RFP). A non-limiting list of fluorescent or other markers includes proteins detectable by luminescence (e.g. luciferase) or colorimetric assay (e.g. GUS). A review of new classes of fluorescent proteins and applications can be found in Trends in Biochemical Sciences [Rodriguez, Erik A.; Campbell, Robert E.; Lin, John Y.; Lin, Michael Z.; Miyawaki, Atsushi; Palmer, Amy E.; Shu. Xiaokun; Zhang, Jin; Tsien, Roger Y. “The Growing and Glowing Toolbox of Fluorescent and Photoactive Proteins”. Trends in Biochemical Sciences. doi:10.1016j.tibs.2016.09.010].

Alternatively, marker-less transformation can be used to obtain organisms e.g. plants without mentioned marker genes, the techniques for which are known in the art.

According to one embodiment, introducing the exogenous polynucleotide into the at least one cell comprises subjecting the at least one cell to genome editing using artificially engineered nucleases.

Various exemplary methods may be used to introduce nucleic acid alterations to a gene of interest and various agents may be used for implementing same according to specific embodiments of the present invention.

For example, genome editing may be carried out using engineered endonucleases. These include the meganucleases, Zinc finger nucleases (ZFNs), transcription-activator like effector nucleases (TALENs) and CRISPR/Cas system. Such methods are discussed in detail in U.S. Patent Application No. 20190085038 and in PCT publication no. WO 2019058253, both incorporated herein by reference in their entirety. Additional methods which may be used include e.g. the “Hit and run” or “in-out” method, “double-replacement” or “tag and exchange” strategy, Site-Specific Recombinases, and Transposase systems, as discussed in U.S. Patent Application No. 20190085038, incorporated herein by reference in its entirety.

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

According to one embodiment, there is provided a method of producing a plant having a terpene synthase activity of interest, the method comprising: (a) crossing a plant which comprises a polynucleotide sequence encoding a polypeptide having a terpene synthase activity, the polypeptide comprising an amino acid sequence selected from the group consisting of SEQ ID NO: 1-45 with a plant of interest, the plant of interest being a recurrent parent; and (b) selecting from a progeny of the crossing a plant having the terpene synthase activity of interest.

According to one embodiment, there is provided a method of producing a plant having a terpene profile of interest, the method comprising: (a) crossing the plant of some embodiments of the invention, with a plant of interest; and (b) selecting from a progeny of the crossing a plant having the terpene profile of interest.

According to one embodiment, there is provided a method of producing a plant having a transporter activity of interest, the method comprising: (a) crossing the plant of some embodiments of the invention, with a plant of interest; and (b) selecting from a progeny of the crossing a plant having the transporter activity of interest.

According to one embodiment, the method comprises selecting for progeny of the crossing a plant having the terpene synthase activity of interest and the transporter activity of interest.

According to one embodiment, the method comprises selecting for progeny of the crossing a plant having the terpene profile of interest and the transporter activity of interest.

According to one embodiment, the method further comprises backcrossing to the plant of interest (e.g. recurrent plant).

According to one embodiment, selection of a plant (or other organism e.g. non-human organism) comprising an introgression is effected genotypically, e.g. by presence or expression of a gene or lack of presence or expression (e.g. marker assisted breeding).

According to one embodiment, selection of progeny plant (or other organism e.g. non-human organism) having the desired characteristics is performed by analyzing cells (e.g. plant cells) for a marker, e.g. molecular genetic marker, i.e. an indicator that is used in methods for visualizing differences in nucleic acid sequences. Examples of such indicators are restriction fragment length polymorphism (RFLP) markers, amplified fragment length polymorphism (AFLP) markers, single nucleotide polymorphisms (SNPs), insertion/deletion (INDEL) mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location.

Methods for detecting sequence alteration are well known in the art and include, but are not limited to, DNA and RNA sequencing (e.g., next generation sequencing), electrophoresis, an enzyme-based mismatch detection assay and a hybridization assay such as PCR, RT-PCR, RNase protection, in-situ hybridization, primer extension, Southern blot, Northern Blot and dot blot analysis. Various methods used for detection of single nucleotide polymorphisms (SNPs) can also be used, such as PCR based T7 endonuclease, Heteroduplex and Sanger sequencing, or PCR followed by restriction digest to detect appearance or disappearance of unique restriction site/s.

According to one embodiment, selection of a plant comprising an introgression is effected phenotypically.

According to one embodiment, selection of plant (or other organism e.g. non-human organism) having a terpene profile is effected using any method known in the art, e.g. by gas chromatograph (GC), thin layer chromatography (TLC), gas chromatography coupled to mass spectrometer (GC-MS), liquid chromatography (LC), ion mobility spectrometers (IMS), high-field ion mobility spectrometers asymmetric waveform (FAIMS, high-field asymmetric waveform ion mobility spectrometry) electro-chemical sensors, electrochemical sensor arrays and colorimetric sensors.

An exemplary method for assessing a terpene profile includes the following steps: (1) breaking one or more cell (e.g. plant cell) to release its chemical constituents; (2) extracting the sample using a suitable solvent (or through distillation or the trapping of compounds); (3) separating the desired terpene from other undesired contents of the extracts that confound analysis and quantification; and (4) using appropriate method of analysis (e.g. TLC, GC, or LC), as discussed in detail in Jiang et al., Curr Protoc Plant Biol. (2016) 1: 345-358.

According to one embodiment, selection of plant (or other organism e.g. non-human organism) having a transporter activity is effected using any method known in the art, e.g. using assays designed to measure transporter function, for example, ATP hydrolysis, conformational change, or solute transport, such as biophysical methods with different fluorescent dyes (as discussed in detail in Bartosiewicza and Krasowskab, Zeitschrift fur Naturforschung 2009) or positron emission tomography (PET) imaging of the plant.

According to one embodiment, a phenotype is determined prior to a genotype.

According to one embodiment, a genotype is determined prior to a phenotype.

Regardless of the method of introduction, the present teachings provide for an isolated cell (e.g., plant cell or bacterial cell) which comprises an exogenous nucleic acid sequence encoding the polypeptide of some embodiments of the invention.

According to one embodiment, the present teachings provide for an isolated cell (e.g., plant cell or bacterial cell) or organism (e.g. non-human organism, e.g. plant or part thereof, bacteria, yeast, insect, etc.) co-expressing a polypeptide having a terpene synthase activity and a polypeptide having a transporter activity.

According to one embodiment, the present teachings provide for an isolated cell (e.g., plant cell or bacterial cell) or organism (e.g. non-human organism, e.g. plant or part thereof, bacteria, yeast, insect, etc.) co-expressing polypeptides having two separate terpene synthase activities.

According to one embodiment, the present teachings provide for an isolated cell (e.g., plant cell or bacterial cell) or organism (e.g. non-human organism, e.g. plant or part thereof, bacteria, yeast, insect, etc.) co-expressing polypeptides having two separate transporter activities.

The present invention further provides methods of producing a plant comprising sowing the seed of some embodiments of the invention or planting a plantlet of the plant of some embodiments of the invention under conditions which allow growth of the plant.

The present invention further provides methods of producing a terpene of interest, the method comprising recovering a terpene fraction comprising the terpene of interest from the organism (e.g. non-human organism e.g. plant, yeast, bacteria, insect, etc.) of some embodiments of the invention.

According to one embodiment, the terpene is extracted as oil (e.g. essential oil).

The term “oil” refers to a mixture of compounds obtained from the extraction of cannabis plants. Such compounds include, but are not limited to, cannabinoids, terpenes, terpenoids, and other compounds found in the plant. The exact composition of oil will depend on the plant (e.g. strain of cannabis) that is used for extraction, the efficiency and process of the extraction itself, and any additives that might be incorporated to alter the palatability or improve administration of the oil.

The term oil includes derivatives thereof, including racemic mixtures, enantiomers, diastereomers, hydrates, salts, solvates, metabolites, analogs, and homologs. Composition, production and plant families of oils comprising terpenes (e.g. essential oils), are described in detail in Kirk-Othmer Encyclopedia of Chemical Technology, 4^{th} Edition S. Price, Aromatherapy Workbook—Understanding Essential Oils from Plant to Bottle, (HarperCollins Publishers, 1993; J. Rose, The Aromatherapy Book—Applications & Inhalations (North Atlantic Books, 1992); and in The Merck Index, 13^{th} Edition, each of which is incorporated herein by reference.

According to one embodiment, the terpene comprising oil is extracted (harvested, recovered, etc.) from a plant (e.g. trichome-bearing plant) using any of several known suitable methods, including but not limited to steam distillation, organic extraction, and microwave techniques. The various chemical components of the oil may be isolated through traditional organic extraction and purification methods. Further, the plant and its essential oil may be subjected to qualitative and quantitative analysis using any method known in the art. The composition and quality of the oil may be determined using, for example, gas chromatography/mass spectroscopy (GC/MS).

For example, leaves can be directly (without prior freezing) steam-distilled and solvent-extracted using, for example, pentane in a condenser-cooled Likens-Nickerson apparatus (as discussed in Ringer et al., 2003). Terpenes and other components can then be identified by comparison of retention times and mass spectra to those of authentic standards in gas chromatography with mass spectrometry detection. Quantification can be achieved by gas chromatography with flame ionization detection based upon calibration curves with known amounts of authentic standards and normalization to the peak area of camphor as internal standard.

Methods for extraction of oil from cannabis plants are well known in the art, and include for example, CO₂ extraction, alcohol (e.g., ethanol) extraction, and oil extraction, as discussed in PCT publication no. WO 2016123475, incorporated herein by reference in its entirety.

Methods of extraction of terpenes are well known in the art, and include for example, solvent extraction with organic solvents, solid phase microextraction (SPME), headspace method involving the concentration of compounds by condensation or by the application of solid phase resins/columns.

Any composition obtainable from the organism (e.g. non-human organism e.g. plant, bacteria, yeast, insect, etc.) of some embodiments of the invention (e.g. oil comprising secondary metabolites including terpenes, terpenoids and cannabinoids) can be used in pharmaceutical, cosmetic, cleaning or recreational compositions.

As used herein a “pharmaceutical composition” or “cosmetic composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the composition obtainable from the organism (e.g. non-human organism, e.g. plant, bacteria, yeast, insect, etc.) accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion), molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (e.g. a terpene comprising composition) effective to prevent, alleviate or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

According to one embodiment, a therapeutically effective amount of a composition comprising liquid chromatography pooled fractions of a plant (e.g. cannabis plant) comprising active ingredients (cannabinoids, terpenes and/or terpenoids).

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide appropriate levels of the active ingredient sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to one embodiment, and without being limited to, the pharmaceutical composition may be used for the treatment of pain, inflammation, depression, anxiety, addiction, eating disorder, epilepsy, cancer, and infections (e.g.viral, fungal and bacterial infections).

The preparations made from the organism (e.g. non-human organism, e.g. plants or parts thereof, bacteria, yeast, insect, etc.) of the present invention (e.g. oil comprising secondary metabolites including terpenes, terpenoids and cannabinoids) can be administered to a subject (e.g., a human or animal) in need thereof in a variety of other forms including a nutraceutical composition.

As used herein, a “nutraceutical composition” refers to any substance that may be considered a food or part of a food and provides medical or health benefits, including the prevention and treatment of disease, or ease of disease symptoms. In some embodiments, a nutraceutical composition is intended to supplement the diet (i.e. dietary supplements) and contains at least one or more of the following ingredients: a vitamin; a mineral; an herb; a botanical; a fruit; a vegetable; an amino acid; or a concentrate, metabolite, constituent, or extract of any of the previously mentioned ingredients; and combinations thereof.

In some embodiments, a nutraceutical composition of the present invention can be administered as a “dietary supplement,” as defined by the U.S. Food and Drug Administration, which is a product taken by mouth that contains a “dietary ingredient” such as, but not limited to, a vitamin, a mineral, an herb or other botanical, an amino acid, and substances such as an enzyme, an organ tissue, a glandular, a metabolite, or an extract or concentrate thereof.

Non-limiting forms of nutraceutical compositions of the present invention include: a tablet, a capsule, a pill, a softgel, a gelcap, a liquid, a powder, a solution, a tincture, a suspension, a syrup, an oil, or other forms known to persons of skill in the art. A nutraceutical composition can also be in the form of a food, such as, but not limited to, a food bar, a beverage, a food gel, a food additive/supplement, a powder, a syrup, and combinations thereof.

The preparations made from the organism (e.g. non-human organism, e.g. plants or parts thereof, bacteria, yeast, insect, etc.) of the present invention can be formulated in any of a variety of forms utilized by the cosmetic industry, including but not limited to, creams, lotions, oils, powders, solutions, gels, sprays, ointments, salves, soap, wash, lipsticks, fingernail and toe nail polish, eye and facial makeup, perfumes, aftershaves, manicures, permanent waves, shaving foams and creams, shampoos, conditioners, hair colors, hair sprays and gels, deodorants, baby products, bath oils, bubble baths, bath salts, butters and many other types of products.

The CTFA Cosmetic Ingredient Handbook, Second Edition (1992), incorporated herein by reference, describes a wide variety of non-limiting cosmetic ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyffhizinate), skin treating agents, thickeners, and vitamins and derivatives thereof.

The preparations made from the organism (e.g. non-human organism, e.g. plants or parts thereof, bacteria, yeast, insect, etc.) of the present invention can be formulated in a cleaning composition (e.g. soap, detergent, liquid, etc.) in any of a variety of suitable forms and for use of various purposes (e.g. cleaning of laundry, dishes, household, surfaces, textiles, automobiles, etc.). The Handbook for Cleaning/Decontamination of Surfaces 2007, incorporated herein by reference, describes a wide variety of non-limiting uses of cleaning products and formulations thereof, which are suitable for use in the compositions of the present invention.

In a further aspect the invention, the preparations made from the organism (e.g. non-human organism, e.g. plants or parts thereof, bacteria, yeast, insect, etc.) of the present invention (e.g. oil comprising secondary metabolites including terpenes, terpenoids and cannabinoids) can be used by a subject (e.g. human) for recreational purposes.

In a further aspect the invention, there is provided a food or processed product (e.g., dry, liquid, paste) obtainable from the organism (e.g. non-human organism, e.g. plant, bacteria, yeast, insect, etc.) of some embodiments of the invention. A food or processed product is any ingestible preparation containing the plant, or parts thereof, of the instant invention, or preparations made from these plants or parts thereof (or from the other organisms described herein). Thus, the plants, plant parts, or preparations obtained therefrom (or from the other organisms described herein) are suitable for human (or animal) consumption. Also provided are feed products adapted for animal consumption.

The food or processed product of the present invention can also include additional additives such as, for example, sweeteners, flavorings, colors, preservatives, nutritive additives such as vitamins and minerals, condiments, amino acids (i.e. essential amino acids), emulsifiers, pH control agents such as acidulants, hydrocolloids, antifoams and release agents, flour improving or strengthening agents, raising or leavening agents, cohesive agents, gases and chelating agents, the utility and effects of which are well-known in the art. See Merriani-Websters Collegiate Dictionary, 10th Edition, 1993.

According to a specific embodiment, the food or processed product obtainable from the organism (e.g. non-human organism, e.g. plant, bacteria, yeast, insect, etc.) of some embodiments of the invention is an oil.

According to one embodiment, the oil is an essential oil.

According to specific embodiments the oil is a cannabis oil.

According to a specific embodiment, the food or processed product (e.g. oil) comprises a DNA of the organism (e.g. non-human organism, e.g. plant, bacteria, yeast, insect, etc.).

It is expected that during the life of a patent maturing from this application many relevant terpene synthases and transporters will be developed and the scope of the term “terpene synthase” and “transporter” is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 58 is expressed in a DNA sequence format (e.g., reciting T for thymine), but it can refer to either a DNA sequence that corresponds to a terpene synthase nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes 1-111 Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074: 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Procedures

Preparation of Enriched Glandular Capitate-Stalked Trichomes

The capitate-stalked trichome enrichment was conducted using a modified BeadBeater™ and fine mesh procedure previously described [Wu T et al., PLoS ONE (2012) 7(8): e41822].

Glandular trichomes were isolated from C. sativa plants. Mature female inflorescences were placed into a 350 ml bead beater chamber (BioSpec Products, Inc., Bartlesville, Okla., USA). The chamber was filled with 80-100 g of 0.5 mm diameter glass beads, 1% PVP soluble 360,000 MW and 0.6% Methylcellulose and ice-cold gland wash buffer (50 mM Tris-ICL, pH 7.5, 200 mM Sorbitol, 20 mM Sucrose, 10 mM KCl, 5 mM MgCl₂, 0.5 mM HK₂PO₄, 5 mM succininc acid, 1 mM EGTA, 1 mM Aurintricarboxylic acid, 14 mM b-nercaptoethanol). The BeadBeater was turned on for 5 minutes.

Following the bead beater procedure, the trichomes were separated from the plant material and glass beads by passing the supernatant of the chamber through a 350-μm sieve. The residual plant material and beads were rinsed twice with ice-cold gland wash buffer and passed again through the 350-μm sieve. The combined filtrates (with ice-cold gland wash buffer) were then consecutively filtered through 150-μm, 105-μm, 80-μm and 50-μm sieves. The resulting filtrate was collected and centrifuged for 2.5 min at 2500 rpm. Subsequently, the supernatant was decanted and the pellet was stored in −80° C. The purity of the purified trichome fraction was confirmed by inspecting the samples using a light microscope.

RNA Extraction Method

RNA was extracted from the aforementioned isolated trichomes and 6-week old female inflorescences of four different varieties of Cannabis sativa. RNA extraction was conducted using a poly-A based strategy of mRNA purification [Spectrum™ Plant Total RNA Kit (SIGMA-ALDRICH)]. The isolated trichomes and inflorescences underwent the initial immersion in lysis buffer as following. 50 mg of isolated trichomes were suspended in lysis solution, vortexed for 30 seconds, and inserted for 5 minutes in an ice-cold desiccator. 50 mg of inflorescences were suspended in lysis solution and vortexed for 30 seconds. Both fractions were subsequently treated as described in the Spectrum™ Plant Total RNA Kit.

The RNA samples were analyzed for gene expression following preparation of RNAseq libraries and high throughput sequencing via Illumina technology. The RNA library preparation was conducted using a TruSeq mRNA RNAseq library preparation kit (Illumina).

The Statistics Ofthe RNAseq Results

Raw reads were filtered and cleaned using Trimmomatic [Bolger, A. M., et al. Bioinformatics. (2014) 30(15)-2114-20] to remove adapters and the FASTX-Toolkit version 0.0.13.2 for (1) trimming read-end nucleotides with quality scores <30 using fastq_quality_trimmer and (2) removing reads with <70% base pairs with quality score ≤30 using fastq_quality_filter. TopHat2 [Trapnell C. et al., Nat Biotechnol. (2010) 28:511) was used to map the clean reads onto the cannabis genome reference (A physical and genetic map of Cannabis sativa identifies extensive rearrangements at the THC/CBD acid synthase loci. Laverty K. U., et al. Genome Res. (2019) 29(1):146-156]. The genomic version used for gene analysis and prediction based on the RNAseq data was GCA_000230575.3_ASM23057v3_genomic, downloaded from NCBI: ftp://ftp(dot)ncbi(dot)nlm(dot)nih(dot)gov/genomes/all/GCA/000/230/575/GCA_000230 575.3_ASM23057v3/GCA_000230575.3_ASM23057v3_genomic.fna.gz.

Expression of Terpene Synthases Genes

Putative terpene synthase genes were synthesized by the TWIST Co., and cloned into Novagen pET-28a(+) with a 6×His tag at its' N-terminus. The recombinant plasmid was then sequenced for authentication and transformed into BL21(DE3) pLysS and pLysE strains of E. coli (Agilent Technologies) for heterologous expression. An overnight culture was used to inoculate a 20-200 mL culture, induced with 0.1 mM IPTG at an OD 600 nm >0,8 for initiation of recombinant protein translation then incubated at 18° C. and 250 RPM overnight. The cells were collected by centrifugation and the recombinant enzymes were extracted either as a crude extract or purified on a Ni-NTA agarose resin as previously described [Gonda I. et al., The Plant Journal (2013) 61, 458-472].

Terpene Synthases Activity Assay

To determine the terpene synthase catalytic activity, reaction mixtures containing 100 μl of protein extract or purified protein were incubated overnight at 30° C. with either 10 μM geranyl diphosphate (GPP) or farnesyl diphosphate (FPP), in a final volume of 200 μl using a buffer containing 50 mM bis-tris, pH 6.9, 1 mM DTT, 10% (v/v) glycerol, 0.1 mM of MnCl2 and 10 mM of MgCl2. The volatiles produced were extracted by solid-phase microextraction (SPME) according to the methodology previously described [Iijima Y. et al., Plant Physiol (2004) 136: 3724-3736]. As controls, reactions were carried out without the cofactors MnCl2 and MgCl2, or protein extracts were heat inactivated by incubation at 100° C. for 5 minutes, or a short peptide product of an empty pET100 vector was utilized.

For solid-phase microextraction (SPME) analysis, a 57298-U SPME fiber assembly Divinylbenzene/Carboxen/Polydimethylsiloxane (DVB/CAR/PDMS, Supelco) needle size 23 ga, StableFlex, was used with an autosampler and analyzed by GC-MS as previously described [Davidovich-Rikanati R. et al., Nature Biotech (2007) 25:899-901; Davidovich-Rikanati R. et al., Plant J (2008) 56:228-238]. The SPME fiber were adsorbed for 30 min. by automatic HS-SPME at 50° C. followed by injection of the fiber into a GC-MS injection port for 5 min. (splitless), for desorption of the volatiles. Chiral separation was conducted on a Restek Rt™-bDEXsm column (30 m length 0.25 mm i.d., 0.25 μm film thickness, 2,3-di-O-methyl-6-O-tert-butyl dimethylsilyl beta cyclodextrin added into 14% cyanopropylphenyl/86% dimethyl polysiloxane). Helium (0.8 ml min-1) was used as a carrier gas with splitless injection. The injector temperature was 250° C., and the detector temperature was 230° C. The following conditions were used: initial temperature 40° C. for 5 min., followed by a ramp of 2° C./min. to 110° C., and 10° C./min up to 230° C. (5 min.). A quadrupole mass detector with electron ionization at 70 eV was used to acquire the MS data in the range of 41 to 350 m/z. The identification of the volatiles was assigned by comparison spectral data with the W10N11 and HPCH2205 GC-MS libraries and with authentic standard when available.

Selection of ABC and PTR Transporter Genes

A list of the Cannabis sativa ABC and PTR transporter genes was prepared based on the available Cannabis genomes (www(dot)medicinalgenomics(dot)com, and genome(dot)ccbr(dot)utoronto(dot)ca/FAQ/). GCA_000230575.3_ASM23057v3_genomic was downloaded from NCBI: ftp(dot)ncbi(dot)nlm(dot)nih(dot)gov/genomes/all/GCA/000/230/575/GCA_000230575.3_ASM23057v3/GCA_000230575.3_ASM23057v3_genomic.fna.gz and the gene expression data based on the RNA-seq analysis described above for the members of the transporter family was prepared. Genes with the highest level of enrichment in the highly-purified trichome fraction were selected for functional expression. Six ABC and PTR-transporter genes expressing the highest enrichment were selected, and their complete cDNA sequence was determined based on the RNA-seq sequencing. In addition to these six genes, homologous genes sharing adjacent genomic proximity (tandem genes) to these genes were chosen, totaling 12 selected genes.

Expression of ABC and PTR Transporter Genes in Yeast and Tobacco

The genes were expressed in yeast and tobacco. For yeast, the ABC and PTR Transporter genes were synthesized by the TWIST Co, cloned and expressed (individually) in the yeast expression vector pESC-URA (Agilent Technologies) for expression under the yeast native gal-10 promoter. Competent Saccharomyces cerevisiae (strain Y1HGold, CloneTech) cells were prepared, transformed by each of the resulting vector and selected on yeast synthetic media lacking Uracil as previously described [GietzR. D., Methods Mol. Biol. (2014) 1205:1-12].

For tobacco, the ABC and PTR Transporter genes were cloned into a plant binary expression vector (pGreen 0029) via Agrobacterium mediated transformation as previously described [Cohen, S. et at, Nat. Commun. (2014) 5:4026]. These genes were cloned between the cauliflower mosaic virus (CaMl,) 35S promoter and the polyadenylation signal from the nopaline synthase gene and inserted into tobacco BY2 cells and plants (var. Samson) via stable transformation..

Evaluation of the Cannabis ABC and PTR Transporter's Toxicity Reduction Effect of Terpenes and Cannabinoids in Yeast

The rescue from terpene and cannabinoid toxicity in cell culture is assessed by analyzing the growth inhibition effect of various monoterpenes or cannabinoids on a matrix of yeast cells harboring an empty vector, vectors expressing ABC or PTR Transporters or wild-type cells. The Terpene and Cannabinoid toxic effect is assessed based on a procedure consisting plate assays as previously described [Demissie Z. A. et al., Planta (2018) 10.1007/s00425-018-3064-x].

Yeast cells are grown to OD600 of approximately 1.0, streaked on YPD plate, followed by placing three filter disks per plate at equal distances. The filter disks are infused with 20 μl of DMSO containing various concentrations of Terpenes and Cannabinoids and incubated at 30° C. for 72 hours. The inhibition zone is calculated by measuring the diameter of the yeast clear area around the filter disk, and the toxic effect is correlated accordingly.

An alternative method is to measure the yeast growth as a function of turbidity. Transformed yeast cells were grown on URA-glucose broth at 30° C. at 200 RPM, and after 24 hours 100 μl was used to inoculate 5 ml of URA-galactose broth in the same conditions till O.D.600 of 1.5. The yeast was further diluted to OD600=0.05 with 10 ml URA-galactose, and divided into two 5 ml aliquots into separate 50 ml falcon tubes. The first falcon (control) was added 78 μl acetonitrile (the solvent that the CBD was dissolved in), and the second one was added 78 μl CBD solution in acetonitrile (final CBD concentration=0.5 mM). The samples were incubated for 2-8 hours in 30° C. at 250 RPM, and their growth/turbidity at OD600 was assessed in intervals of one hour.

Measurement of Uptake of CBD into Yeast and BY-2 Cells

An additional strategy for determining transporter function is to measure the amount of exogenous CBD taken up and incorporated into the cells.

Yeast Cells

Transformed yeast cells were grown on URA-glucose broth at 30° C. at 200 RPM, and after 24 hours 100 μl was used to inoculate 5 ml of URA-galactose broth in the same conditions till OD₆₀₀ of 1.5 ml. The yeast was incubated with 0.5 mM CBD for 2-8 hours in 30° C. at 100 RPM, then centrifuged at 15000 g for 1 minute, and washed twice with 1 ml distilled water. The pellet was weighed and underwent HPLC analysis to determine the pellet's CBD content. The pellet was extracted in 1 ml methanol, and following centrifugation and filter was analyzed by HPLC using a diode-array detector and CBD was quantified against a commercial standard.

BY2 Tobacco Cells

2.5 ml of newly diluted transformed BY2 cells were incubated with 0.5 mM CBD for 8 hours in 25° C. at 100 RPM, then centrifuged at 15000 g for 1 minute, and washed twice with 1 ml distilled water. The pellet was weighed and underwent HPLC analysis to determine the pellet CBD content, as indicated above.

Measurement of Uptake of Terpenes into Yeast

Transformed yeast cells were grown on URA-glucose broth at 30° C. at 200 RPM, and after 24 hours 100 μl was used to inoculate 5 ml of URA-galactose broth in the same conditions till OD600 of 1.5. 1.5 ml of the yeast was incubated with limonene (0.2 mM), caryophyllene (0.2 mM) or alpha-pinene (0.3 mM) for 2 hours in 30° C. at 100 RPM, then centrifuged at 15000 g for 1 minute, and washed twice with 1 ml distilled water. The pellet was weighed and extracted in 1 ml MTBE, underwent an overnight vortex, and following centrifugation and filter was analyzed by GC-MS as for the analysis of terpenes following extraction by solid-phase microextraction (SPME) according to the methodology previously described [Iijima Y. et al., Plant Physiol (2004) 136: 3724-3736] and the terpenes were quantified against a commercial standard.

Evaluation of the Cannabis ABC and PTR Transporter's Toxicity Reduction Effect of Terpenes and Cannabinoids in Tobacco Cells

The toxic effect and cell death activity of terpenes and cannabinoids in tobacco was assessed on cell cultures as follows: 7-day-old suspension cultured BY-2 tobacco cells at a 5-fold dilution in logarithmic growth phase, incubated in Gamborg B5 medium, was incubated in the presence of 0.5 mM CBD for 48 hours as previously described [Sirikantaramas S. et al., Plant Cell Physiol. (2005) 46:1578-1582].

Cell death in the samples was evaluated using the cell viability indicator fluorescein diacetate (FDA, Sigma-Aldrich). Cell suspension (2.5 mL) was stained with 50 μL of 0.5% fluorescein diacetate (FDA) dissolved in acetone for 10 min. The cells were rinsed three times with I mL isotonic medium to remove excess staining solution. Viability was assessed immediately using an Olympus BX50 fluorescence microscope equipped with a suitable barrier filter (excitation 460-490 nm and emission 510 nm). FDA staining of viable cells results in bright green fluorescence, while nonviable cells remain colorless.

### Example 1

### Identifying Terpene Synthases from Cannabis

45 full length terpene synthase genes of the a, b and g terpene synthase families were identified and established as the TPS used herein (see Table 1, below).

TABLE 1
Amino acid sequences of the 45 identified gene products

| Terpene Synthase Gene | | |
|---|---|---|
| (according to chromosomal | Amino Acid | Nucleic Acid |
| position) | Sequence | Sequence |
| 790-1 | SEQ ID NO: 1 | SEQ ID NO: 58 |
| 790-2 | SEQ ID NO: 2 | SEQ ID NO: 59 |
| 790-3 | SEQ ID NO: 3 | SEQ ID NO: 60 |
| 790-4 | SEQ ID NO: 4 | SEQ ID NO: 61 |
| 790-5 | SEQ ID NO: 5 | SEQ ID NO: 62 |
| 792-1 | SEQ ID NO: 6 | SEQ ID NO: 63 |
| 792-2 | SEQ ID NO: 7 | SEQ ID NO: 64 |
| 792-3 | SEQ ID NO: 8 | SEQ ID NO: 65 |
| 792-4 | SEQ ID NO: 9 | SEQ ID NO: 66 |
| 792-5 | SEQ ID NO: 10 | SEQ ID NO: 67 |
| 792-6 | SEQ ID NO: 11 | SEQ ID NO: 68 |
| 792-7 | SEQ ID NO: 12 | SEQ ID NO: 69 |
| 792-8 | SEQ ID NO: 13 | SEQ ID NO: 70 |
| 792-9 | SEQ ID NO: 14 | SEQ ID NO: 71 |
| 793-1 | SEQ ID NO: 15 | SEQ ID NO: 72 |
| 793-2 | SEQ ID NO: 16 | SEQ ID NO: 73 |
| 794-1 | SEQ ID NO: 17 | SEQ ID NO: 74 |
| 795-1 | SEQ ID NO: 18 | SEQ ID NO: 75 |
| 795-2 | SEQ ID NO: 19 | SEQ ID NO: 76 |
| 795-3 | SEQ ID NO: 20 | SEQ ID NO: 77 |
| 796-1 | SEQ ID NO: 21 | SEQ ID NO: 78 |
| 796-2 | SEQ ID NO: 22 | SEQ ID NO: 79 |
| 796-3 | SEQ ID NO: 23 | SEQ ID NO: 80 |
| 796-4 | SEQ ID NO: 24 | SEQ ID NO: 81 |
| 796-5 | SEQ ID NO: 25 | SEQ ID NO: 82 |
| 796-7 | SEQ ID NO: 26 | SEQ ID NO: 83 |
| 796-8 | SEQ ID NO: 27 | SEQ ID NO: 84 |
| 798-1 | SEQ ID NO: 28 | SEQ ID NO: 85 |
| 798-2 | SEQ ID NO: 29 | SEQ ID NO: 86 |
| 799-1 | SEQ ID NO: 30 | SEQ ID NO: 87 |
| 799-2 | SEQ ID NO: 31 | SEQ ID NO: 88 |
| 799-3 | SEQ ID NO: 32 | SEQ ID NO: 89 |
| 799-4 | SEQ ID NO: 33 | SEQ ID NO: 90 |
| 799-5 | SEQ ID NO: 34 | SEQ ID NO: 91 |
| 799-5a | SEQ ID NO: 35 | SEQ ID NO: 92 |
| AG046-1 | SEQ ID NO: 36 | SEQ ID NO: 93 |
| AG1075-1 | SEQ ID NO: 37 | SEQ ID NO: 94 |
| AG2522-1 | SEQ ID NO: 38 | SEQ ID NO: 95 |
| AG3403-1 | SEQ ID NO: 39 | SEQ ID NO: 96 |
| AG3403-2 | SEQ ID NO: 40 | SEQ ID NO: 97 |
| AG3908-1 | SEQ ID NO: 41 | SEQ ID NO: 98 |
| AG4076-1 | SEQ ID NO: 42 | SEQ ID NO: 99 |
| AG7638-1 | SEQ ID NO: 43 | SEQ ID NO: 100 |
| S600-1 | SEQ ID NO: 44 | SEQ ID NO: 101 |
| S1328-1 | SEQ ID NO: 45 | SEQ ID NO: 102 |

The genes were named following their chromosomal position (e.g. 792-1 for chromosome named 790 to 799, and the relative positions of each gene on the chromosome), or presence on a small scaffold not yet placed on any of the 10 chromosomes (e.g., AGXXX, Sxxx) as shown in Table 2. Thus, for example, the genes on chromosome 6 were termed 796-1, 796-2, 796-3, 796-4, 796-5, 796-7, and 796-8. Generally, the closely spaced genes are the result of evolutionary tandem duplications and thus have highly similar sequences.

Sequence alignments allowed for the classification of the TPS into predicted cyclic monoterpene synthases (family b), non-cyclic monoterpene synthases (family g), and sesquiterpene synthases (family a). The genomic coordinates of the individual genes are listed in Table 2 (below) and serve to characterize the introgression that may be followed in a breeding program.

TABLE 2
Genomic coordinates of the cannabis TPS genes

| Gene | Chromosome | Start bp | End bp |
|---|---|---|---|
| (new name) | (contig) | (genome start bp) | (genome end bp) |
| 790-1 | CM010790.1 | 2632226 | 2640191 |
| 790-2 | CM010790.1 | 2644932 | 2649253 |
| 790-3 | CM010790.1 | 3181246 | 3195135 |
| 790-4 | CM010790.1 | 3232611 | 3238021 |
| 790-5 | CM010790.1 | 3264188 | 3269148 |
| 792-1 | CM010792.1 | 820621 | 824457 |
| 792-2 | CM010792.1 | 1903370 | 1907666 |
| 792-3 | CM010792.1 | 1918918 | 1924206 |
| 792-4 | CM010792.1 | 9022466 | 9025748 |
| 792-5 | CM010792.1 | 9357211 | 9361536 |
| 792-6 | CM010792.1 | 9627543 | 9629928 |
| 792-7 | CM010792.1 | 57117886 | 57123968 |
| 792-8 | CM010792.1 | 75105984 | 75111432 |
| 792-9 | CM010792.1 | 75111190 | 75137262 |
| 793-1 | CM010793.1 | 43922210 | 43933552 |
| 793-2 | CM010793.1 | 43957596 | 43963179 |
| 794-1 | CM010794.1 | 1010638 | 1018837 |
| 795-1 | CM010795.2 | 17856832 | 17859188 |
| 795-2 | CM010795.2 | 17877618 | 17880153 |
| 795-3 | CM010795.2 | 17913211 | 17920796 |
| 796-1 | CM010796.2 | 10493362 | 10496651 |
| 796-2 | CM010796.2 | 10518048 | 10521638 |
| 796-3 | CM010796.2 | 10534390 | 10537972 |
| 796-4 | CM010796.2 | 38322420 | 38325367 |
| 796-5 | CM010796.2 | 52162308 | 52166849 |
| 796-7 | CM010796.2 | 52202797 | 52206934 |
| 796-8 | CM010796.2 | 52211106 | 52216657 |
| 798-1 | CM010798.2 | 32376874 | 32385064 |
| 798-2 | CM010798.2 | 40593521 | 40596699 |
| 799-1 | CM010799.2 | 22504768 | 22509388 |
| 799-2 | CM010799.2 | 22528277 | 22531870 |
| 799-3 | CM010799.2 | 22546241 | 22559267 |
| 799-4 | CM010799.2 | 47131972 | 47138311 |
| 799-5 | CM010799.2 | 47141564 | 47156143 |
| 799-5a | CM010799.2 | 47141564 | 47156143 |
| AG046-1 | AGQN03000461.1 | 3304 | 5576 |
| AG1075-1 | AGQN03001075.1 | 25596 | 39369 |
| AG2522-1 | AGQN03002522.1 | 98042 | 108660 |
| AG3403-1 | AGQN03003403.1 | 13043 | 20959 |
| AG3403-2 | AGQN03003403.1 | 25690 | 37016 |
| AG3908-1 | AGQN03003908.1 | 1499 | 8204 |
| AG4076-1 | AGQN03004076.1 | 40892 | 48479 |
| AG7638-1 | AGQN03007638.1 | 535 | 5572 |

The closest reported homolog of each of the 45 genes, as identified by BLAST of the NCBI database is shown in Table 3.

TABLE 3
Homologs of each of the TPS genes

| Gene name | Closest homolog | Closest organism | % identity |
|---|---|---|---|
| 790-1 | ARE72258 | Cannabis sativa | 89 |
| 790-2 | ARE72259 | Cannabis sativa | 97 |
| 790-3 | ARE72257 | Cannabis sativa | 92 |
| 790-4 | A7IZZ1 | Cannabis sativa | 99 |
| 790-5 | ARE72255 | Cannabis sativa | 100 |
| 792-1 | ARE72252 | Cannabis sativa | 94 |
| 792-2 | ARE72252 | Cannabis sativa | 96 |
| 792-3 | ARE72260 | Cannabis sativa | 99 |
| 792-4 | ACI32640.1 | Humulus lupulus | 75 |
| 792-5 | ACI32640.1 | Humulus lupulus | 75 |
| 792-6 | XP-015902224.1 | Ziziphus jujuba | 49 |
| 792-7 | XP-015902224.1 | Ziziphus jujuba | 51 |
| 792-8 | XP-015902224.1 | Ziziphus jujuba | 51 |
| 792-9 | XP-015902224.1 | Ziziphus jujuba | 50 |
| 793-1 | ARE72250 | Cannabis sativa | 82 |
| 793-2 | ARE72252 | Cannabis sativa | 76 |
| 794-1 | ARE72260 | Cannabis sativa | 66 |
| 795-1 | ARE72256 | Cannabis sativa | 74 |
| 795-2 | ARE72256 | Cannabis sativa | 72 |
| 795-3 | ARE72256 | Cannabis sativa | 71 |
| 796-1 | XP-015886715.1 | Ziziphus jujuba | 59 |
| 796-2 | XP-010098578.1 | Morus notabilis | 60 |
| 796-3 | XP-010088087.1 | Morus notabilis | 61 |
| 796-4 | CBI20483.3 | Vitis vinifera | 61 |
| 796-5 | ACI32640.1 | Humulus hipulus | 75 |
| 796-7 | ACI32640.1 | Humulus lupulus | 75 |
| 796-8 | ACI32639.1 | Humulus lupulus | 75 |
| 798-1 | ARE72256 | Cannabis sativa | 96 |
| 798-2 | XP-010109558.1 | Morus notabilis | 65 |
| 799-1 | ARE72252 | Cannabis sativa | 95 |
| 799-2 | ARE72250 | Cannabis sativa | 99 |
| 799-3 | ARE72252 | Cannabis sativa | 89 |
| 799-4 | ARE72252 | Cannabis sativa | 91 |
| 799-5 | ARE72250 | Cannabis sativa | 98 |
| 799-5a | ARE72250 | Cannabis sativa | 98 |
| AG0461-1 | ARE72256 | Cannabis sativa | 72 |
| AG1075-1 | ARE72254 | Cannabis sativa | 79 |
| AG2522-1 | ARE72251 | Cannabis sativa | 99 |
| AG3403-1 | ARE72258 | Cannabis sativa | 89 |
| AG3403-2 | ARE72259 | Cannabis sativa | 97 |
| AG3908-1 | ARE72254 | Cannabis sativa | 91 |
| AG4076-1 | ARE72256 | Cannabis sativa | 97 |
| AG7638-1 | ARE72254 | Cannabis sativa | 91 |
| S600-1 | ARE72252 | Cannabis sativa | 77 |
| S1328-1 | PON82650 | Trema orientale | 73 |

Of note, % identity of each protein sequence was identified by BLASTP. Nearest homolog not identified by species binomial is from Cannabis sativa species.

In order to determine which members of the TPS are preferentially expressed in the inflorescence trichomes, and which are associated with non-trichome cells, gene expression was compared between whole inflorescence tissue and a partially purified trichome fraction derived from inflorescence tissue. As the terpene production is thought to be restricted to the Cannabis trichomes, a differential expression analysis of plant tissues consisting of a trichome purification gradient was conducted.

Gene expression of the enriched trichome fraction was compared to that of the whole female inflorescence. RNA was extracted from ca 6-week old inflorescences of four different varieties of Cannabis sativa used for medicinal purposes in Israel (referred to herein as Var CSI1, CS12, CS13, CS14). In addition, the enriched trichome fraction of the inflorescence from variety CS14 was extracted for RNA using the same RNA extraction method.

The statistics of the RNAseq results are presented in Table 4, below.

TABLE 4
The statistics of the RNAseq results

| | | | | Percent mapping vs. |
|---|---|---|---|---|
| | | | | Genome (indicates |
| | | | | % reads successfully |
| | No. | No. | No. | identified as homologous |
| Sample | raw-reads | clean-reads | clean-reads | to the Cannabis genome) |
| CS11 | 11323706 | 10919965 | 96.43455 | 84.4 |
| inflorescence | | | | |
| CS12 | 9522705 | 9165590 | 96.24986 | 86.3 |
| inflorescence | | | | |
| CS13 | 10377699 | 10017573 | 96.52981 | 88.2 |
| inflorescence | | | | |
| CS14 | 10606649 | 10222341 | 96.37673 | 88 |
| inflorescence | | | | |
| CS14 | 21734921 | 20915436 | 96.2 | 83.0 |
| trichomes | | | | |

Table 5 (below) shows the gene expression results (FPKM, Fragments Per Kilobase Million) for the 45 TPS genes for the inflorescences of the four varieties, as well as for the enriched trichome fraction of Var CS14. Clear differences in specific gene expression were observed between the inflorescences of the four varieties. Thus, for example, terpene synthase 790-4 is expressed strongly in Var CS13 and based on the trichome enrichment in Var CS14 this gene is enriched 11.5 fold in the trichomes. Thus, among the 4 tested varieties, Var CS13 would be a good source in breeding for cannabis varieties containing the terpenes produced by this particular terpene synthase. Of the 45 TPS genes present in the genome, the expression of 21 were particularly enriched in the trichome fraction (more than 3×) of the only trichome-enriched variety (CS14). Since the whole inflorescence comprises the trichomes, there were no genes that were expressed only in the enriched trichome sample.

TABLE 5
Gene expression of the Cannabis TPS genes in 4 varieties inflorescences
and the enriched trichome fraction of Var CS14
(Numbers indicate FPKM values)

| | Var CS11- | Var CS12- | Var CS13- | Var CS14- | | |
|---|---|---|---|---|---|---|
| Gene | whole | whole | whole | whole | trichome | fold |
| name | inflorescence | inflorescence | inflorescence | inflorescence | Var CS14 | enrichment |
| 790-1 | 7.1 | 7.8 | 6.8 | 6.1 | 36.5 | 6.0 |
| 790-2 | 1.4 | 1.1 | 2.4 | 1.4 | 2.6 | 1.8 |
| 790-3 | 24.0 | 21.0 | 10.0 | 5.5 | 43.5 | 7.9 |
| 790-4 | 22.0 | 47.0 | 100.0 | 13.0 | 150.0 | 11.5 |
| 790-5 | 1.3 | 4.2 | 2.0 | 1.5 | 33.0 | 22.0 |
| 792-1 | 2.7 | 1.9 | 1.0 | 1.9 | 1.3 | 0.7 |
| 792-2 | 8.8 | 5.6 | 3.6 | 3.2 | 20.5 | 6.4 |
| 792-3 | 19.0 | 14.0 | 8.6 | 9.6 | 16.0 | 1.7 |
| 792-4 | 0.0 | 0.0 | 0.0 | 1.6 | 1.1 | 0.7 |
| 792-5 | 1.0 | 1.0 | 0.0 | 1.1 | 1.0 | 0.9 |
| 792-6 | 0.0 | 0.0 | 0.0 | 1.7 | 0.8 | 0.4 |
| 792-7 | 11.0 | 29.0 | 18.0 | 9.9 | 165.0 | 16.7 |
| 792-8 | 10.0 | 9.0 | 6.3 | 7.6 | 15.5 | 2.0 |
| 792-9 | 4.7 | 9.8 | 11.0 | 13.0 | 14.0 | 1.1 |
| 793-1 | 3.1 | 6.9 | 2.4 | 4.3 | 27.0 | 6.3 |
| 793-2 | 5.3 | 1.6 | 6.8 | 1.9 | 8.2 | 4.3 |
| 794-1 | 27.0 | 2.0 | 1.4 | 1.8 | 1.7 | 0.9 |
| 795-1 | 1.2 | 2.6 | 1.0 | 3.3 | 14.9 | 4.5 |
| 795-2 | 1.2 | 2.9 | 0.0 | 3.4 | 21.0 | 6.2 |
| 795-3 | 1.2 | 2.1 | 0.0 | 3.3 | 9.2 | 2.8 |
| 796-1 | 2.4 | 2.8 | 2.7 | 2.0 | 9.8 | 4.9 |
| 796-2 | 4.5 | 1.8 | 4.8 | 1.1 | 2.4 | 2.1 |
| 796-3 | 3.9 | 2.0 | 0.0 | 2.2 | 3.3 | 1.5 |
| 796-4 | 1.2 | 1.0 | 0.0 | 1.0 | 2.6 | 2.6 |
| 796-5 | 1.3 | 0.0 | 1.6 | 0.0 | 0.0 | 0.0 |
| 796-7 | 1.5 | 1.0 | 1.2 | 2.1 | 0.0 | 0.0 |
| 796-8 | 1.2 | 1.1 | 1.8 | 1.6 | 1.0 | 0.6 |
| 798-1 | 10.0 | 12.0 | 3.6 | 5.7 | 42.5 | 7.5 |
| 798-2 | 1.8 | 1.7 | 1.7 | 1.8 | 1.9 | 1.0 |
| 799-1 | 11.0 | 7.4 | 3.0 | 4.6 | 21.5 | 4.7 |
| 799-2 | 22.0 | 7.3 | 7.7 | 3.7 | 36.0 | 9.7 |
| 799-3 | 22.0 | 8.3 | 2.5 | 4.0 | 32.5 | 8.1 |
| 799-4 | 21.0 | 7.8 | 2.4 | 3.4 | 37.5 | 11.0 |
| 799-5 | 8.7 | 6.9 | 6.3 | 3.2 | 30.0 | 9.4 |
| AG0461-1 | 1.0 | 2.5 | 0.0 | 4.6 | 16.0 | 3.5 |
| AG1075-1 | 3.1 | 3.6 | 2.5 | 2.7 | 4.8 | 1.8 |
| AG2522-1 | 1.8 | 1.2 | 12.0 | 1.2 | 2.1 | 1.8 |
| AG3403-1 | 8.4 | 8.5 | 5.8 | 6.9 | 42.0 | 6.1 |
| AG3403-2 | 3.0 | 2.1 | 2.1 | 2.1 | 11.4 | 5.4 |
| AG3908-1 | 1.5 | 1.0 | 1.3 | 1.3 | 1.1 | 0.8 |
| AG4076-1 | 2.6 | 14.0 | 2.3 | 5.8 | 61.0 | 10.5 |
| AG7638-1 | 1.4 | 1.0 | 1.0 | 1.3 | 1.5 | 1.1 |

Since the trichomes are considered as the cells in which terpene synthesis as well as cannabinoid synthesis takes place, these results strongly support the roles of the identified genes in the synthesis of the trichome-specific terpenes of the Cannabis varieties. Reciprocally, those genes not showing enrichment of expression in the purified trichome preparation presumably are responsible for the volatile terpene composition of the cannabis tissues not enriched in trichomes, which also have use in cannabis consumption.

### Example 2

### Functional Expression of the Terpene Synthase Genes

The present inventors characterized the biochemical function of the individual genes. Putative terpene synthases were functionally expressed in bacteria and their enzymatic products identified using methods previously disclosed [Davidovich-Rikanati R. et al., Nature Biotech (2007) 25:899-901; Davidovich-Rikanati R. et al., Plant J (2008) 56:228-238; Gonda I. et al., The Plant Journal (2013) 61, 458-472; Iijima Y. et al., Plant Physiol (2004) 136: 3724-3736] and described in the ‘general materials and experimental procedures’ section above.

The functional expression of the 45 genes identified them as accountable for the synthesis of monoterpenes and sesquiterpenes, as illustrated for example, for the gene product of 792-3 (see Table 6, below).

TABLE 6
The functional expression of the terpene synthase genes

| TPS | Monoterpenes produced | Sesquiterpenes produced |
|---|---|---|
| expressed | with GPP | with FPP |
| 790-1 | (Z)-beta-ocimene and (E)- | |
| | beta-ocimene | |
| 790-2 | (Z)-beta-ocimene and (E)- | farnesene<(E,E)-alpha-> and |
| | beta-ocimene | unidentified sesquiterpene |
| 790-4 | D-limonene, alpha-pinene, | |
| | beta pinene | |
| 792-3 | | caryophyllene, humulene |
| 792-7 | beta-myrcene/geraniol | |
| 794-1 | | selina-3,7(11)-diene and |
| | | additional unidentified |
| | | sesquiterpenes |
| 795-1 | geraniol | curcumene, bisabolene, |
| | | bisabolol and several |
| | | additional unidentified |
| | | sesquiterpenes |
| 796-3 | geraniol | |
| 799-2 | beta-myrcene/ | gamma and delta-selinene and |
| | D-limonene/nerol | additional 10 unidentified |
| | | sesquiterpenes |
| 799-4 | myrcene | beta and alpha selinenes and 4 |
| | | additional unidentified |
| | | sesquiterpenes |
| AG2522-1 | beta-myrcene and | caryophyllene 9 epi, |
| | linalool | aromadendrene, |
| | | bicyclogermacrene and 10 |
| | | additional unidentified |
| | | sesquiterpenes |
| AG3403-2 | beta-myrcene and geraniol | |
| AG3908-1 | eucalyptol, gamma | |
| | terpinene, alpha thujene | |
| | and 6 additional monoterpenes | |
| AG046-1 | | bisabolene |

Accordingly, the biochemical function and terpene products of individual gene sequences are identified and the particular sequence is linked to its volatile terpene product. Thus, the gene sequences of the 45 members of the Cannabis terpene synthase family described are attributed to particular terpene compounds.

Accordingly, the differences in gene expression of particular genes among the different varieties, combined with differences in gene sequences due to variety-specific allelic polymorphisms for individual genes, allow for the selection of genetically defined varieties based on their terpene synthase sequences and gene expression. These are used for the breeding of novel cannabis varieties with desirable volatile composition, using a marker-assisted genetic breeding program based on the variety-specific gene sequences.

### Example 3

### Breeding Cannabis Varieties

Cannabis varieties expressing desired volatile terpene components are bred with cannabis varieties for generation of new cannabis varieties comprising a desirable volatile composition. Breeding is carried out via backcrossing, marker assisted breeding, selfing, or via genetic transformation (e.g. via genetic transfer of chromosomal segments from select varieties chosen on the basis of their TPS gene expression pattern).

### Example 4

### Identifying Transporters in Cannabis sativa

Twelve full length ATP-binding cassette transporter (ABC) and Peptide Transporter (PTR) gene families were identified (see Table 7, below).

TABLE 7
Amino acid sequences of the 12 ABC +
PTR Transporter identified gene products

| Transporter Gene (according | Amino Acid | Nucleic Acid |
|---|---|---|
| to chromosomal position) | Sequence | Sequence |
| T-790-1 | SEQ ID NO: 46 | SEQ ID NO: 103 |
| T-792-1 | SEQ ID NO: 47 | SEQ ID NO: 104 |
| T-792-2 | SEQ ID NO: 48 | SEQ ID NO: 105 |
| T-795-1 | SEQ ID NO: 49 | SEQ ID NO: 106 |
| T-796-1 | SEQ ID NO: 50 | SEQ ID NO: 107 |
| T-796-2 | SEQ ID NO: 51 | SEQ ID NO: 108 |
| T-796-3 | SEQ ID NO: 52 | SEQ ID NO: 109 |
| T-AG0100-1 | SEQ ID NO: 53 | SEQ ID NO: 110 |
| T-AG0100-2 | SEQ ID NO: 54 | SEQ ID NO: 111 |
| T-AG2575-1 | SEQ ID NO: 55 | SEQ ID NO: 112 |
| T-AG2575-2 | SEQ ID NO: 56 | SEQ ID NO: 113 |
| T-AG9876-1 | SEQ ID NO: 57 | SEQ ID NO: 114 |

The ABC+PTR Transporter genes were named following their chromosomal position in the same manner as the Terpene Synthase genes. The Transporter genes were treated and named as a distinct group, and their designated name contains the letter “T” at the beginning of their name (e.g. T-790-1) to differentiate them from the Terpene Synthase genes (e.g. 790-1).

TABLE 8
Genomic coordinates of 12 cannabis
(ABC + PTR) Transporter genes

| Gene | | Start bp | End bp | |
|---|---|---|---|---|
| (new | Chromosome | (genome | (genome | Transporter |
| name) | (contig) | start bp) | end bp) | family |
| T-790-1 | CM010790.1 | 78188811 | 78198320 | ABC-B |
| T-792-1 | CM010792.1 | 10359393 | 10366510 | ABC-G |
| T-792-2 | CM010792.2 | 27429206 | 27441008 | ABC-G |
| T-795-1 | CM010795.2 | 33651495 | 33657701 | ABC-G |
| T-796-1 | CM010796.2 | 2290332 | 2296582 | ABC-G |
| T-796-2 | CM010796.2 | 2297673 | 2303864 | ABC-G |
| T-796-3 | CM010796.2 | 2324151 | 2331474 | ABC-G |
| T-AG0100-1 | AGQN03000100.1 | 177381 | 180571 | PTR/NRT |
| T-AG0100-2 | AGQN03000100.1 | 187363 | 191535 | PTR/NRT |
| T-AG2575-1 | AGQN03002575.1 | 43585 | 54754 | ABC-G |
| T-AG2575-2 | AGQN03002575.1 | 59076 | 67714 | ABC-G |
| T-AG9876-1 | AGQN03009876.1 | 5231 | 10059 | ABC-G |

TABLE 9
Homologs of each of the ABC + PTR Transporter genes

| Gene name | Closest homolog | Closest organism | % identity |
|---|---|---|---|
| T-790-1 | PON87450.1 | Trema orientale | 93 |
| T-792-1 | PON89636.1 | Trema orientale | 76 |
| T-792-2 | PON89636.1 | Trema orientale | 73 |
| T-795-1 | PON89425.1 | Trema orientale | 82 |
| T-796-1 | PON89430.1 | Trema orientale | 81 |
| T-796-2 | PON89425.1 | Trema orientale | 85 |
| T-796-3 | PON89425.1 | Trema orientale | 83 |
| T-AG0100-1 | PON99177.1 | Trema orientale | 79 |
| T-AG0100-2 | PON99176.1 | Trema orientale | 81 |
| T-AG2575-1 | PON66387.1 | Parasponia andersonii | 78 |
| T-AG2575-2 | PON89425.1 | Trema orientale | 78 |
| T-AG9876-1 | PON89425.1 | Trema orientale | 78 |

TABLE 10
Gene expression of 12 Cannabis ABC + PTR Transporter genes in inflorescences of 4 varieties
and the enriched trichome fraction of Var CS14 (Numbers indicate FPKM values)

| | Var CS11- | Var CS12- | Var CS13- | Var CS14- | | |
|---|---|---|---|---|---|---|
| | whole | whole | whole | whole | trichome | fold |
| Gene name | inflorescence | inflorescence | inflorescence | inflorescence | Var CS14 | enrichment |
| T-790-1 | 21 | 32 | 36 | 23 | 185 | 8.0 |
| T-792-1 | 3.6 | 25 | 9.4 | 5.6 | 69 | 12.3 |
| T-792-2 | 9 | 11 | 10 | 3.9 | 22 | 5.6 |
| T-795-1 | 8.5 | 5.9 | 3.9 | 21 | 3 | 0.2 |
| T-796-1 | 76 | 23 | 8.5 | 18 | 7 | 0.4 |
| T-796-2 | 74 | 22 | 9.5 | 21 | 6 | 0.3 |
| T-796-3 | 4.5 | 3.9 | 4 | 3.2 | 10 | 3.2 |
| T-AG0100-1 | 13 | 13 | 39 | 6.7 | 77 | 11.5 |
| T-AG0100-2 | 2.5 | 2.8 | 3.6 | 3 | 3 | 1.2 |
| T-AG2575-1 | 9.8 | 12 | 14 | 5.8 | 57 | 9.7 |
| T-AG2575-2 | 14 | 26 | 17 | 15 | 91 | 6.0 |
| T-AG9876-1 | 11 | 11 | 19 | 5.3 | 61 | 11.4 |

### Example 5

### Evaluation of the Cannabis ABC and PTR Transporter's Toxicity Reduction Effect of Terpenes and Cannabinoids in Yeast and Tobacco Cells

To functionally identify the Cannabis ABC and PTR Transporters, the present inventors took advantage of the toxicity of terpenes to yeast cells and cannabinoids to tobacco BY-2 cells [Demissie Z. A. el al., Planta (2018) 10.1007/s00425-018-3064-x; Sirikantaramas S. el al., Plant Cell Physiol. (2005) 46:1578-1582]. The rescue from terpene and cannabinoid toxicity in cell culture was assessed by analyzing the growth inhibition effect of various monoterpenes or cannabinoids on a matrix of yeast cells harboring an empty vector, vectors expressing ABC or PTR Transporters or wild-type cells. The Terpene and Cannabinoid toxic effect was assessed based on a procedure consisting plate assays as previously described [Demissie Z. A. et al., (2018), supra]. The toxic effect and cell death activity of terpenes and cannabinoids in tobacco was assessed on cell cultures and live plants as previously described [Sirikantaramas S. et al., Plant Cell Physiol. (2005) 46:1578-1582]. Alternatively, as described in the example, effects were assessed based on the growth kinetics of the yeast cells following incubation with the cannabinoid CBD.

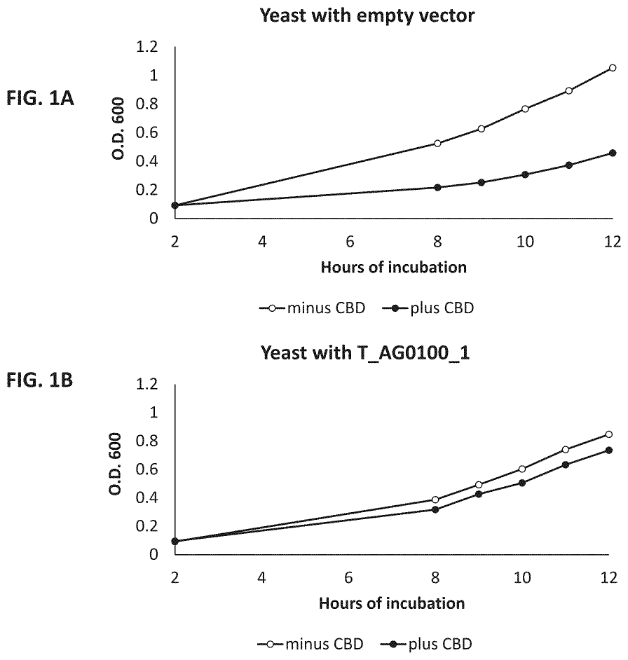

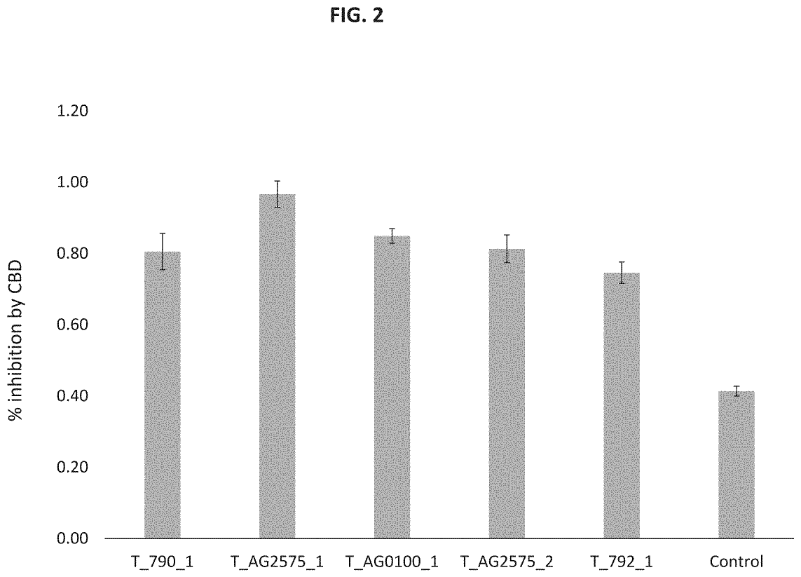

FIGS. 1A-B show the effect of 0.5 mM CBD on growth dynamics of the yeast cells expressing the transporter genes. The figures indicate the time course of yeast growth as expressed in cell turbidity (A600) during the 12 hour period following the initiation of experiment (as described in the ‘general materials and experimental section’ above). It can clearly be seen that the NRT transporter (T-AG0100-1) shows reduced growth inhibition (FIG. 1B), as compared to the control yeast harboring the empty vector (FIG. 1A). FIG. 2 expands these results to members of the B and G family of ABC transporters and shows that from 7-12 hours after initiation of the experiment the control yeast was inhibited by CBD to 42% of its growth without CBD, in comparison to the T-790-1 (ABC-B), T-AG2575-1 (ABC-G), T-AG2575-2 (ABC-G) and T-792-1 (ABC-G), as well as the NRT transporter T-AG0100-1, all of which showed reduced inhibition of growth in response to CBD.

### Example 6

### Evaluation of the Cannabis Transporter on CBD and Terpene Uptake in Yeast and Tobacco Cells

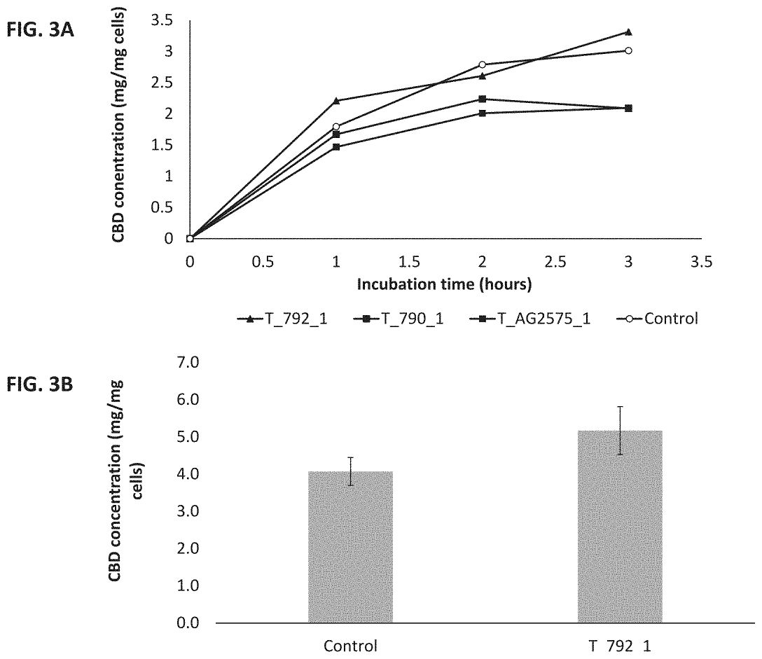

FIG. 3A shows the effect of the ABC transporter genes on uptake of CBD into yeast cells. In this experiment, the transporters T790-1 and T-AG2575-1 showed reduced net uptake of CBD, as compared to the control yeast, indicating a transport function. FIG. 3B further strengthens the results that T-792-1 leads to an increase in CBD levels, compared to the control.

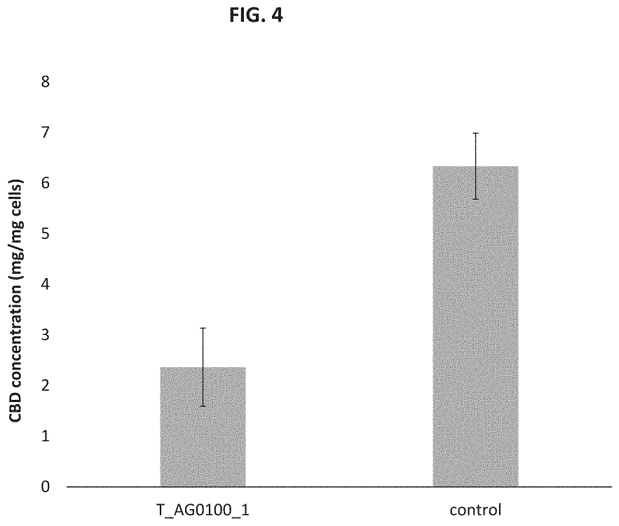

FIG. 4 shows the reduced net uptake of CBD into yeast cells by the NRT transporter AG0100-1 (NRT-yeast cells) as compared to the control, empty plasmid yeast cells following a three hour incubation, again indicating transport function. Results are averages and s.d. of 3 replications.

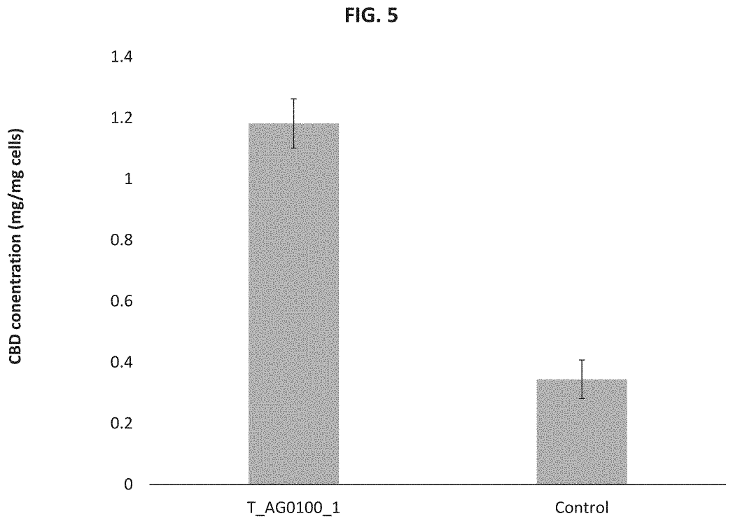

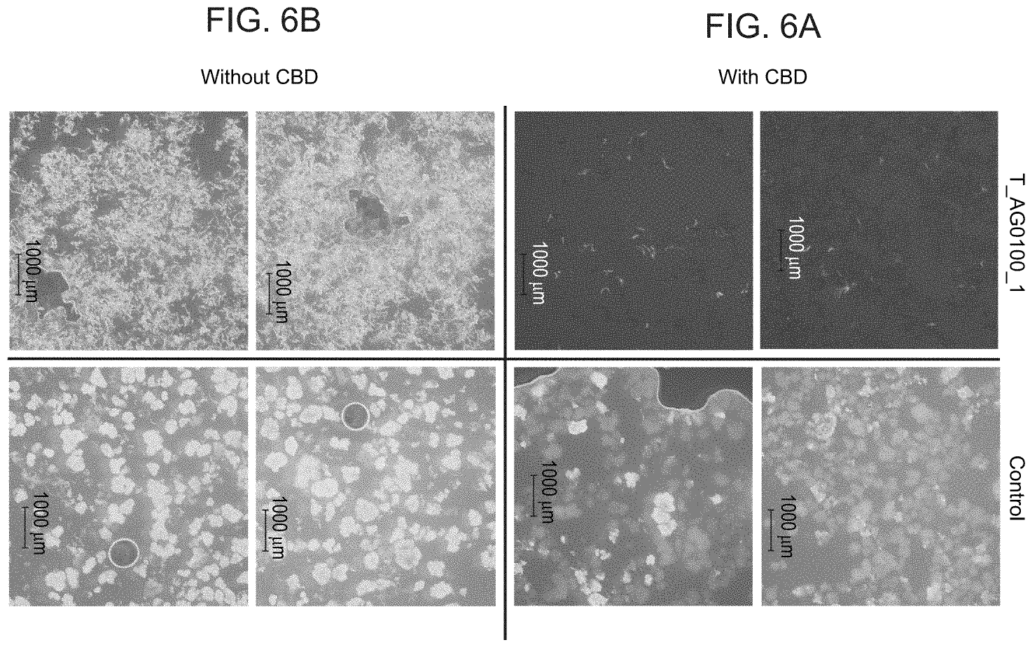

The effect of the NRT transporter gene on CBD uptake in tobacco BY-2 cells is shown in FIG. 5 which shows a strong effect of the NRT gene on the uptake of CBD. A concomitant effect of the NRT transporter on BY-2 cell viability, as indicated by fluorescence using the FDA fluorescence indicator is shown in FIGS. 6A-B.

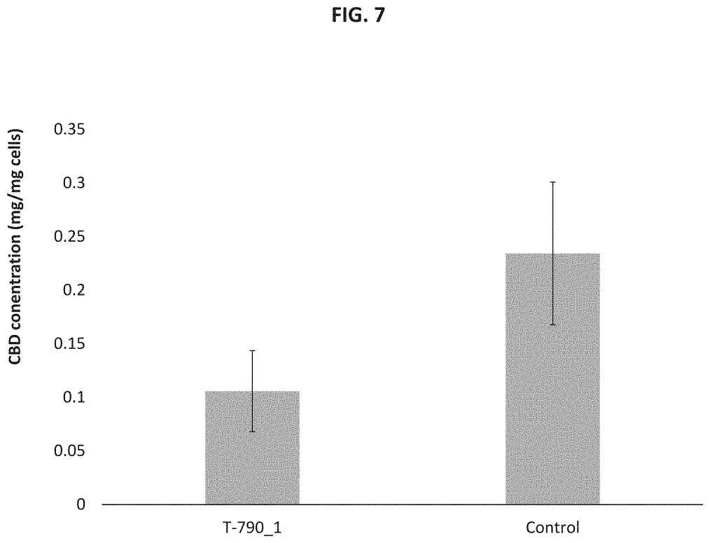

The effect of the T-790-1 ABC B family transporter gene on CBD uptake in tobacco BY-2 cells is shown in FIG. 7 which shows a strong effect of the T-790-1 gene on the uptake of CBD.

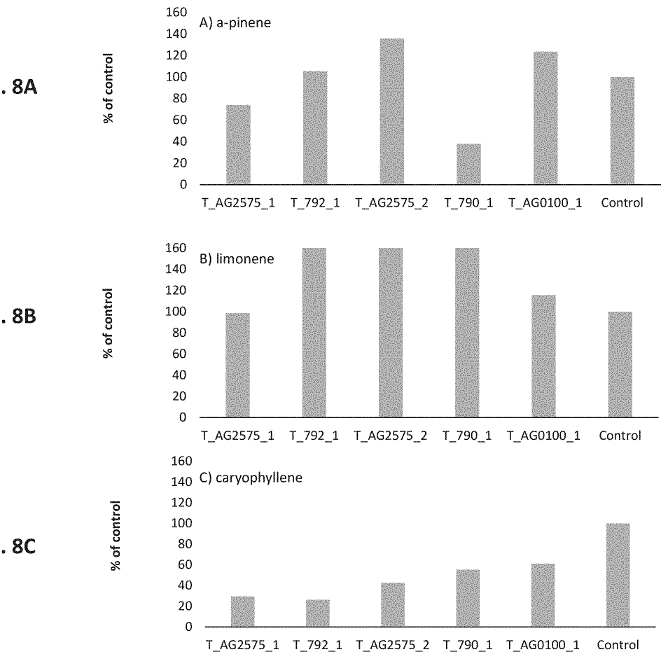

FIGS. 8A-C show the effect of the transporters on terpene uptake by yeast cells. The control yeast are represented as 100% and the effect of each transporter is expressed as percent of control. These results show that the Cannabis transporters reported here have an activity in transporting and modifying the internal yeast levels of the various terpenes.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference.

In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A plant comprising a genome having an introgression which comprises a polynucleotide sequence encoding a polypeptide having a terpene synthase activity, said polypeptide comprising an amino acid sequence selected from the group consisting of SEQ ID NO: 1-45, said introgression comprising allelic variation(s) as compared to a genome of a recurrent parent of the plant.

2. A method of producing a plant having a terpene synthase activity of interest, the method comprising:
(a) crossing a plant which comprises a polynucleotide sequence encoding a polypeptide having a terpene synthase activity, said polypeptide comprising an amino acid sequence selected from the group consisting of SEQ ID NO: 1-45 with a plant of interest, said plant of interest being a recurrent parent; and
(b) selecting from a progeny of said crossing a plant having said terpene synthase activity of interest.

3. (canceled)

4. A method of producing a plant having a terpene profile of interest, the method comprising:
(a) crossing the plant of claim 1, with a plant of interest; and
(b) selecting from a progeny of said crossing a plant having said terpene profile of interest.

5. An organism comprising a genome having been genetically modified to express a polypeptide having a terpene synthase activity, said polypeptide being at least 95% identical to an amino acid sequence selected from the group consisting of SEQ ID NO: 1-45.

6. (canceled)

7. A method of modulating terpene synthesis in an organism, the method comprising over-expressing within at least one cell of the organism a polypeptide having a terpene synthase activity, said polypeptide being at least 95% identical to an amino acid sequence selected from the group consisting of SEQ ID NO: 1-45, thereby modulating terpene synthesis in the organism.

8. An isolated polypeptide comprising an amino acid sequence at least 95% identical to the amino acid sequence selected from the group consisting of SEQ ID NOs: 1-45, wherein the polypeptide, when expressed in an organism, is capable of modulating the synthesis of a terpene of interest.

9. A plant comprising a genome having an introgression which comprises a polynucleotide sequence encoding a polypeptide having a transporter activity, said polypeptide comprising an amino acid sequence selected from the group consisting of SEQ ID NO: 46-57, 116, 118, 120, 122, 124, 126, 128 and 130, said introgression comprising allelic variation(s) as compared to a genome of a recurrent parent of the plant.

10. (canceled)

11. (canceled)

12. A method of producing a plant having a transporter activity of interest, the method comprising:
(a) crossing the plant of claim 9, with a plant of interest; and
(b) selecting from a progeny of said crossing a plant having said transporter activity of interest.

13. (canceled)

14. (canceled)

15. (canceled)

16. An organism comprising a genome having been genetically modified to express a polypeptide having a transporter activity, said polypeptide being at least 95% identical to an amino acid sequence selected from the group consisting of SEQ ID NO: 46-57, 116, 118, 120, 122, 124, 126, 128 and 130.

17. (canceled)

18. A method of modulating transport of metabolites in an organism, the method comprising over-expressing within at least one cell of the organism a polypeptide having a transporter activity, said polypeptide being at least 95% identical to an amino acid sequence selected from the group consisting of SEQ ID NO: 46-57, 116, 118, 120, 122, 124, 126, 128 and 130, thereby modulating the transport of the metabolites in the organism.

19. (canceled)

20. (canceled)

21. (canceled)

22. An isolated polypeptide comprising an amino acid sequence at least 95% identical to the amino acid sequence selected from the group consisting of SEQ ID NOs: 46-57, 116, 118, 120, 122, 124, 126, 128 and 130, wherein the polypeptide, when expressed in an organism, is capable of modulating transport of metabolites.

23. (canceled)

24. (canceled)

25. An isolated polynucleotide encoding a polypeptide having a terpene synthase activity, said polypeptide being at least 95% identical to an amino acid sequence selected from the group consisting of SEQ ID NO: 1-45.

26. An isolated polynucleotide encoding a polypeptide having a transporter activity, said polypeptide being at least 95% identical to an amino acid sequence selected from the group consisting of SEQ ID NO: 46-57, 116, 118, 120, 122, 124, 126, 128 and 130.

27. A nucleic acid construct comprising a nucleic acid sequence of the polynucleotide of claim 25, and a cis-acting regulatory element for directing expression of said nucleic acid sequence in a cell.

28. (canceled)

29. (canceled)

30. (canceled)

31. An isolated cell comprising at least one exogenous polynucleotide according to claim 25.

32. A genetically modified organism comprising at least one exogenous polynucleotide according to claim 25.

33. A plant generated according to the method of claim 2.

34. An organism generated according to the method of claim 7.

35. A method of producing a terpene of interest, the method comprising recovering a terpene fraction comprising the terpene of interest from the plant of claim 1.

36. A terpene containing fraction of the plant of claim 1.

37. (canceled)

38. (canceled)

39. (canceled)

40. A seed of the plant of claim 1.

41. (canceled)

42. A method of producing a plant comprising sowing the seed of claim 40 under conditions which allow growth of the plant.

43. (canceled)

44. (canceled)

45. (canceled)

46. (canceled)

47. (canceled)

48. A pharmaceutical or cosmetic composition, or food or processed product, obtainable from the plant of claim 1.

49. (canceled)

50. (canceled)
